(19) United States
(12) Patent Application Publication
(10) Pub. No.: US 2026/0110012 A1
(43) Pub. Date: Apr. 23, 2026

(54) COMPOSITIONS AND METHODS FOR ENZYMATIC PRODUCTION OF POLYPHENOL AGLYCONE COMPOUNDS

(71) Applicant: SHREENIKA PIONEERING INC., San Diego, CA (US)

(72) Inventors: Jose A. AMAYA, San Diego, CA (US); Jeffrey EHRHARDT, San Diego, CA (US); Jingyi LI, San Diego, CA (US); Drew Taylor WAGNER, San Diego, CA (US)

(21) Appl. No.: 19/361,768

(22) Filed: Oct. 17, 2025

Related U.S. Application Data
(60) Provisional application No. 63/708,836, filed on Oct. 18, 2024.

(51) Int. Cl. C12P 19/46; C12N 9/24; C12P 19/14

(52) U.S. Cl. CPC C12P 19/46; C12N 9/2402; C12P 19/14

(57) ABSTRACT

The present disclosure provides compositions comprising a glycoside hydrolase and one or both of hesperidin and hesperetin, wherein an active site of the glycoside hydrolase comprises first, second, third, fourth, and fifth regions as described herein. The present disclosure also relates to methods for producing a polyphenol aglycone, comprising contacting a phenolic glycoside with a glycoside hydrolase described herein. Also provided are host cells and heterologous expression systems capable of expressing a heterologous glycoside hydrolase, and nucleic acids encoding the glycoside hydrolase.

### SEQUENCE LISTING

The instant application contains a Sequence Listing which has been submitted electronically in XML format and is hereby incorporated by reference in its entirety. Said Sequence Listing XML, created on Oct. 13, 2025, is named “0300-0003WO1_SL.xml” and is 14,925 bytes in size. The sequence listing contained in this .XML file is part of the specification and is hereby incorporated by reference herein in its entirety.

### FIELD OF THE INVENTION

The present disclosure provides compositions comprising a glycoside hydrolase and one or both of hesperidin and hesperetin, wherein an active site of the glycoside hydrolase comprises first, second, third, fourth, and fifth regions as described herein. The present disclosure also relates to methods for producing a polyphenol aglycone, comprising contacting a phenolic glycoside with a glycoside hydrolase described herein. Also provided are host cells and heterologous expression systems capable of expressing a heterologous glycoside hydrolase, and nucleic acids encoding the glycoside hydrolase.

### BACKGROUND

Polyphenol aglycones include flavanone compounds found in citrus fruits, such as naringenin, eriodictyol, and hesperetin, and may have important biological activities, including antioxidant, antibacterial, anti-inflammatory, and cardioprotective effects. These compounds may also be used in the food and beverage industry as flavor-altering, bitter-masking, and/or de-bittering agents. See, e.g., Salehi et al., Pharmaceuticals (Basel) (2019) 12 (1): 11; Deng et al., Evid Based Complement Alternat Med (2020) 2020:6671352; Salehi et al., Int J Mol Sci (2019) 20 (6): 2305; and Wdowiak et al., Nutrients (2022) 14 (13): 2647.

### SUMMARY OF THE INVENTION

In some embodiments, the present disclosure provides a composition comprising a glycoside hydrolase and one or both of hesperidin and hesperetin, wherein an active site of the glycoside hydrolase comprises: a first region comprising at least 80% identity to HQEAII (SEQ ID NO:1); a second region comprising at least 80% identity to DIHSLPGGLNGMGLGE (SEQ ID NO:2): a third region comprising at least 80% identity to PINEPVDNRDITKFGTP (SEQ ID NO:3): a fourth region comprising at least 80% identity to FRPVDYWAKHFAASTNIVFDVHNYYFAGRPT (SEQ ID NO: 4); and a fifth region comprising at least 80% identity to GSAYWTWKFFGNVPVDGEGTQGDYWNY (SEQ ID NO:5).

In some embodiments, the first region comprises at least 90% sequence identity to SEQ ID NO: 1: the second region comprises at least 90% sequence identity to SEQ ID NO:2: the third region comprises at least 90% sequence identity to SEQ ID NO:3: the fourth region comprises at least 90% sequence identity to SEQ ID NO:4: the fifth region comprises at least 90% sequence identity to SEQ ID NO: 5; or any combination thereof. In some embodiments, each of the first, second, third, fourth, and fifth regions include SEQ ID NOs: 1, 2, 3, 4, and 5, respectively.

In some embodiments, the active site of the glycoside hydrolase further comprises a sixth region comprising at least 80% identity to KFPVFVGEWSIQAA (SEQ ID NO:6). In some embodiments, the sixth region comprises at least 80% identity to CSDAKXXXXXXXXKFPVFVGEWSIQAA (SEQ ID NO:7). In some embodiments, the sixth region comprises at least 90% identity to SEQ ID NO:6 or SEQ ID NO:7. In some embodiments, the sixth region comprises SEQ ID NO:6 or SEQ ID NO:7.

In some embodiments, the glycoside hydrolase comprises at least 60% sequence identity to any one of SEQ ID NOs: 9-12. In some embodiments, the glycoside hydrolase comprises at least 70% sequence identity to any one of SEQ ID NOs: 9-12. In some embodiments, the glycoside hydrolase comprises at least 80% sequence identity to any one of SEQ ID NOs: 9-12. In some embodiments, the glycoside hydrolase comprises at least 90% sequence identity to any one of SEQ ID NOs: 9-12. In some embodiments, the glycoside hydrolase comprises any one of SEQ ID NOs: 9-12.

In some embodiments, the composition further comprises DMSO, a surfactant, or both. In some embodiments, the surfactant comprises TRITON X-100, TWEEN, or a combination thereof. In some embodiments, the glycoside hydrolase is capable of hydrolyzing the hesperidin. In some embodiments, the glycoside hydrolase is solubilized in an aqueous solution and the hesperidin and the hesperetin are in solid form.

In some embodiments, the present disclosure provides a method of producing a polyphenol aglycone, comprising contacting a phenolic glycoside with a glycoside hydrolase, wherein an active site of the glycoside hydrolase comprises: a first region comprising at least 80% identity to SEQ ID NO: 1: a second region comprising at least 80% identity to SEQ ID NO:2: a third region comprising at least 80% identity to SEQ ID NO:3: a fourth region comprising at least 80% identity to SEQ ID NO: 4; and a fifth region comprising at least 80% identity to SEQ ID NO:5, wherein the glycoside hydrolase hydrolyzes the phenolic glycoside to form the polyphenol aglycone.

In some embodiments, the glycoside hydrolase is solubilized in an aqueous solution, and the phenolic glycoside is in solid form upon contacting with the glycoside hydrolase.

In some embodiments, the phenolic glycoside comprises a structure of Formula I:

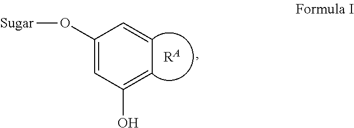

wherein the sugar is a monosaccharide or a disaccharide, and RA is cycloalkyl or heterocyclyl optionally substituted with one or more oxo, alkyl, alkoxyl, alkenyl, alkynyl, hydroxyl, cycloalkyl, heterocyclyl, aryl, or heteroaryl, wherein the cycloalkyl, heterocyclyl, aryl, or heteroaryl is unsubstituted or substituted with one or more oxo, alkyl, alkoxyl, alkenyl, alkynyl, or hydroxyl. In some embodiments, the phenolic glycoside is hesperidin and the polyphenol aglycone is hesperetin.

In some embodiments, the first region comprises at least 90% sequence identity to SEQ ID NO: 1: the second region comprises at least 90% sequence identity to SEQ ID NO:2: the third region comprises at least 90% sequence identity to SEQ ID NO:3: the fourth region comprises at least 90% sequence identity to SEQ ID NO:4: the fifth region comprises at least 90% sequence identity to SEQ ID NO: 5; or any combination thereof. In some embodiments, the active site of the glycoside hydrolase further comprises a sixth region comprising at least 80%, at least 90%, or 100% sequence identity to SEQ ID NO:6 or SEQ ID NO:7. In some embodiments, the glycoside hydrolase comprises at least 60%, at least 70%, at least 80%, at least 90%, or 100% sequence identity to any one of SEQ ID NOs: 9-12.

In some embodiments, the contacting is in the presence of DMSO, a surfactant, or both. In some embodiments, the surfactant comprises TRITON X-100, TWEEN, or a combination thereof. In some embodiments, the method is performed for about 6 hours to about 24 hours. In some embodiments, the method is performed at about 40° C. to about 60° C. In some embodiments, the method is performed at about pH 4 to about pH 6. In some embodiments, a percent conversion of the phenolic glycoside to the polyphenol aglycone is at least 40%. In some embodiments, a percent conversion of the phenolic glycoside to the polyphenol aglycone is at least 80%.

In some embodiments, the present disclosure provides a composition comprising a polyphenol aglycone made by the method. In some embodiments, the polyphenol aglycone is hesperetin.

In some embodiments, the present disclosure provides a host cell capable of expressing a heterologous glycoside hydrolase, wherein an active site of the glycoside hydrolase comprises: a first region comprising at least 80% identity to SEQ ID NO: 1: a second region comprising at least 80% identity to SEQ ID NO:2: a third region comprising at least 80% identity to SEQ ID NO:3: a fourth region comprising at least 80% identity to SEQ ID NO:4; and a fifth region comprising at least 80% identity to SEQ ID NO:5, wherein the glycoside hydrolase is capable of hydrolyzing hesperidin to form hesperetin.

In some embodiments, the present disclosure provides an expression system comprising a host cell comprising a heterologous nucleic acid encoding a glycosidic hydrolase, wherein an active site of the glycoside hydrolase comprises: a first region comprising at least 80% identity to a first region comprising at least 80% identity to SEQ ID NO: 1: a second region comprising at least 80% identity to SEQ ID NO:2: a third region comprising at least 80% identity to SEQ ID NO:3; a fourth region comprising at least 80% identity to SEQ ID NO:4; and a fifth region comprising at least 80% identity to SEQ ID NO:5, wherein the glycoside hydrolase is capable of hydrolyzing hesperidin to form hesperetin.

In some embodiments, the heterologous nucleic acid is integrated into a genome of the host cell. In some embodiments, the heterologous nucleic acid is present on a plasmid in the host cell. In some embodiments, the host cell is a fungal cell. In some embodiments, the host cell is Pichia pastoris or Trichoderma reesei.

In some embodiments, the present disclosure provides a nucleic acid encoding a glycosidic hydrolase, wherein an active site of the glycoside hydrolase comprises: a first region comprising at least 80% identity to a first region comprising at least 80% identity to SEQ ID NO: 1: a second region comprising at least 80% identity to SEQ ID NO:2: a third region comprising at least 80% identity to SEQ ID NO:3: a fourth region comprising at least 80% identity to SEQ ID NO:4; and a fifth region comprising at least 80% identity to SEQ ID NO:5, wherein the glycoside hydrolase is capable of hydrolyzing hesperidin to form hesperetin, and wherein the nucleic acid is operably linked to a heterologous regulatory element. In some embodiments, the heterologous regulatory element is a promoter. In some embodiments, the promoter is a constitutive promoter or an inducible promoter. In some embodiments, the nucleic acid is linked at its 5′ end to a sequence encoding an alpha-factor signal sequence. In some embodiments, wherein the alpha-factor signal sequence comprises SEQ ID NO: 13.

In some embodiments, the first region of the glycoside hydrolase of the host cell, expression system, or nucleic acid comprises at least 90% sequence identity to SEQ ID NO: 1: the second region comprises at least 90% sequence identity to SEQ ID NO:2: the third region comprises at least 90% sequence identity to SEQ ID NO:3: the fourth region comprises at least 90% sequence identity to SEQ ID NO: 4: the fifth region comprises at least 90% sequence identity to SEQ ID NO:5; or any combination thereof. In some embodiments, the active site of the glycoside hydrolase further comprises a sixth region comprising at least 80%, at least 90%, or 100% sequence identity to SEQ ID NO: 6 or SEQ ID NO:7. In some embodiments, the glycoside hydrolase comprises at least 60%, at least 70%, at least 80%, at least 90%, or 100% sequence identity to any one of SEQ ID NOs: 9-12.

In some embodiments, the present disclosure provides a vector comprising the nucleic acid described herein. In some embodiments, the vector is a fungal expression vector, optionally a Pichia expression vector or a Trichoderma expression vector.

### BRIEF DESCRIPTION OF THE DRAWINGS

The following drawings form part of the present specification and are included to further demonstrate exemplary embodiments of certain aspects of the present disclosure.

FIG. 1 illustrates the hydrolysis of hesperidin to form hesperetin and rutinose by a glycoside hydrolase.

FIG. 2 shows HPLC results from an experiment to test different co-solvents and surfactant conditions for hesperetin production as described in embodiments herein. The conditions tested were: 0.5% glycyrrhizic acid (top panel): 0.5% TRITON™ X-100 (middle panel); and 10% DMSO (bottom panel). Structures of the surfactants and DMSO are shown in the inset. The first peak around 4.8 min is hesperidin. The second peak around 7.9 min is hesperetin.

FIG. 3 shows results from an experiment comparing hesperetin production from a glycoside hydrolase from Acremonium sp. DSM 24697 as described in Weiz et al., Appl Microbiol Biotechnol (2019) 103:9493-9504 (“AoGH5”) and a glycoside hydrolase as described in embodiments herein (“SEQ ID NO: 12”). The amount (left axis; bars) and percent conversion (right axis; lines) of hesperetin with high or low substrate amounts was measured. HesD=hesperidin; HesT=hesperetin.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within x and y. When describing a number of amino acids “between” two regions (e.g., a first region and a second region of a protein described herein, such as a glycoside hydrolase described herein), the number refers to the number of amino acid residues beginning after the last residue of the first indicated region and ending before the first residue of the second indicated region.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides described herein, e.g., a glycoside hydrolase.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding a glycoside hydrolase, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the polynucleotides described herein.

An “expression vector or vectors” (also referred to as “expression construct”) can be constructed to include one or more protein of interest-encoding nucleic acids (e.g., nucleic acid encoding a glycoside hydrolase described herein) operably linked to expression control sequences functional in a host organism. Expression vectors applicable for use in host organisms include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). In some embodiments, the expression vector comprises a nucleic acid encoding a protein described herein, e.g., a glycoside hydrolase.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., a gene encoding a glycoside hydrolase and an additional gene encoding another enzyme that complements the glycoside hydrolase reaction as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example: for bacterial host cells: pQE vectors (Qiagen), pBluescript plasmids, pNH vectors, pTWIST vectors (TWIST Bioscience), lambda-ZAP vectors (Stratagenc): pTrc99a, pKK223-3, pDR540, and pRIT2T (Pharmacia): for eukaryotic host cells: pPICZ, pPICα, pPIC, pXT1, pSG5 (Stratagene), pSVK3, pBPV, pMSG, and pSVLSV40 (Pharmacia). However, any other plasmid or other vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding a glycoside hydrolase described herein. In some embodiments, the host cell is a bacterial cell, a fungal cell, an algal cell, a cyanobacterial cell, or a plant cell.

A genetic alteration that makes an organism or cell “non-natural” can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous, or both heterologous and homologous polypeptides for the referenced species. Additional modifications include, for example, non-coding regulatory regions in which the modifications alter expression of a gene or operon.

A host cell or host organism capable of expressing or overexpressing a nucleic acid (e.g., a gene) or polypeptide (e.g., an enzyme), or engineered to express or overexpress the nucleic acid or to overexpress the polypeptide, has been genetically engineered (e.g., through recombinant DNA technology) to include a gene or nucleic acid sequence (which may encode the polypeptide) that it does not naturally include, or to express an endogenous gene at a level that exceeds its level of expression in a non-engineered host cell. As non-limiting examples, a host cell or host organism engineered to express or overexpress a nucleic acid or polypeptide can have any modifications that affect a coding sequence of a gene, the position of a gene on a chromosome or episome, or regulatory elements associated with a gene. A gene can also be overexpressed by increasing the copy number of a gene in the host cell or host organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell or host organism engineered to under-express (or to have reduced expression of) a nucleic acid (e.g., a gene) or to under-express a polypeptide (e.g., an enzyme) can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or downregulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a non-naturally occurring nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

The term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host organism. For example, a host cell comprising a heterologous nucleic acid encoding a glycosidic hydrolase includes any host cell in which the heterologous nucleic acid encoding a glycosidic hydrolase is derived from a different species. In some embodiments, the disclosure provide a host cell comprising a heterologous glycosidic hydrolase, which can include a host cell in which the heterologous glycosidic hydrolase is derived from a different species. Accordingly, exogenous expression of an encoding nucleic acid can utilize either or both of a heterologous or homologous encoding nucleic acid. In some embodiments, the disclosure provides a host cell comprising a heterologous nucleic acid encoding a glycosidic hydrolase, e.g., a glycosidic hydrolase comprising a first region, a second region, a third region, a fourth region and a fifth region as described here.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or combinations thereof, and still be considered as more than one exogenous nucleic acid. For example, as described herein, a host cell or host organism can be engineered to express at least two, three, four, five, six, seven, eight, nine, ten or more exogenous nucleic acids encoding desired proteins or enzymes of a particular biosynthesis pathway. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, or integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell or host organism using techniques known in the art including, but not limited to, conjugation, electroporation, chemical transformation, transduction, transfection, and ultrasound transformation. Optionally, for exogenous expression in E. coli or other microbial host cells, some nucleic acid sequences in the genes or cDNAs of eukaryotic nucleic acids can encode targeting signals such as an N-terminal mitochondrial or other targeting signal, which can be removed before transformation into the prokaryotic host cells if desired. For exogenous expression in yeast or other eukaryotic host cells, genes can be expressed in the cytosol without the addition of leader sequence, or can be targeted to mitochondrion or other organelles or for secretion by the addition of a suitable targeting sequence such as a mitochondrial targeting or secretion signal suitable for the host cells. Thus, it is understood that appropriate modifications to a nucleic acid sequence to remove or include a targeting sequence, can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cell or host organism of interest by replacing at least one codon of the native sequence with codons that are more frequently or most frequently used in the genes of that host cell or host organism while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing a glycoside hydrolase described herein.

The terms “peptide.” “polypeptide.” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of a polypeptide is known as the “N-terminus” (and also referred to as the amino-terminus, NH₂-terminus, N-terminal end or amine-terminus), which refers to the free amine (—NH₂) group of the first amino acid residue of the protein or polypeptide. The end of a polypeptide is known as the “C-terminus” (and also referred to as the carboxy-terminus, carboxyl-terminus, C-terminal end, or COOH-terminus), which refers to the free carboxyl group (—COOH) of the last amino acid residue of the protein or polypeptide. Unless otherwise specified, sequences of polypeptides throughout the present disclosure are listed from N-terminus to C-terminus, and sequences of polynucleotides throughout the present disclosure are listed from the 5′ end to the 3′ end.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg. R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra ct al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (Sec), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,” “non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type” or “original” or “natural” such as wild-type strains of the referenced species, or a wild-type protein or nucleic acid sequence. Likewise, amino acids found in polypeptides of the wild type organism can be referred to as “original” or “natural” with regards to any amino acid position.

An “amino acid substitution” refers to a polypeptide that includes one or more substitutions of wild-type or naturally occurring amino acid(s) with a different amino acid relative to the wild-type or naturally occurring amino acid at that particular residue of the polypeptide. The substituted amino acid may be a synthetic or naturally occurring amino acid. In some embodiments, the substituted amino acid is a naturally occurring amino acid, e.g., Ala, Arg, Asn, Asp, Cys, Gln, Glu, Gly, His, Ile, Leu, Lys, Met, Phe, Pro, Ser, Thr, Trp. Tyr, and Val. In some embodiments, the substituted amino acid is an unnatural or synthetic amino acid. Substitution mutants may be described using an abbreviated system. For example, a substitution mutation in which the fifth (5th) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide or nucleic acid is a molecule that has been removed from its natural environment. It is also understood that “isolated” polypeptides or nucleic acids may be included in a composition, e.g., with buffers, stabilizing agents, and/or salts, and still be considered isolated. As used herein, “isolated” does not necessarily imply any particular level purity of the polypeptide or nucleic acid.

The term “recombinant” when used in reference to a nucleic acid or polypeptide means that the nucleic acid or polypeptide results from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acids and polypeptides.

The term “domain” when used in reference to a polypeptide means a distinct functional and/or structural unit in the polypeptide. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

The term “active site” when used in reference to a polypeptide, e.g., an enzyme, refers to one or more regions in the enzyme that is important for function of the enzyme, e.g., catalysis, substrate binding, and/or cofactor binding. The active site may be a single contiguous region of amino acids, or the active site may comprise one or more regions of amino acids, separated by one or more non-active site residues, that are in proximity to each other in the three-dimensional structure of the enzyme. In embodiments where the enzyme is a multimeric structure (e.g., dimer, trimer, including homodimers, heterodimers, homotrimers, heterotrimers, and the like), the active site may be present upon formation of the multimeric structure, e.g., the active site may span the region between two or more monomers.

As used herein, the term “sequence similarity.” (% similarity) refers to the degree of identity or correspondence between nucleic acid or amino acid sequences. In the context of polynucleotides. “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the polypeptide encoded by the polynucleotides.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu: Polar, uncharged side chains: Ser, Thr, Asn, Gln: Hydrophobic side chains: Ala, Val, Ile, Leu, Met, Phe, Tyr, Trp: Other: Cys, Gly, Pro. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or amino acid sequences is determined when sequences are aligned for maximum homology, and generally not including gaps or truncations. Additional sequences added to a polypeptide, including but not limited to immunodetection tags, purification tags, localization sequences (presence or absence), etc., do not affect the % identity unless otherwise specified.

Algorithms known to those skilled in the art, such as Align, BLAST, ClustalW and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleotide sequences.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequences.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping, i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score): sec. e.g., Xu and Zhang (2010), Bioinformatics 26 (7): 889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align, DALI, STRUCTAL, MINRMS, and the like.

The term “alkyl” means an acyclic alkyl moiety that is linear or branched, preferably containing one or more carbon atoms, e.g., about 1 to 20, about 1 to 10, about 1 to 8, about 1 to 6, or about 1 to 4 carbon atoms. Alkyl includes methyl, ethyl, propyl, butyl, pentyl, hexyl, heptyl, octyl, etc. It will be understood by one of ordinary skill in the art that unless otherwise specified, the chemical names throughout the present disclosure include all isomers thereof, e.g., “propyl” includes isopropyl and n-propyl. “butyl” includes isobutyl, tert-butyl, and sec-butyl, and the like.

The term “alkoxyl” includes linear or branched oxy-containing moieties, each having an alkyl portion as described above, e.g., having about 1 to 20, about 1 to 10, about 1 to 8, about 1 to 6, or about 1 to 4 carbon atoms.

The term “alkenyl” refers to an unsaturated, acyclic hydrocarbon moiety that is linear or branched and that contains at least one double bond, e.g., 1, 2, 3, 4, 5, or more than 5 double bonds, and preferably containing about 2 to 20, or 2 to 10, or 2 to 8, or 2 to 6, or 2 to 4 carbon atoms. The term “alkynyl” refers to an unsaturated, acyclic hydrocarbon moiety that is linear or branched and that contains at least one triple bond, e.g., 1, 2, 3, 4, 5, or more than 5 double bonds, and preferably containing about 2 to 20, or 2 to 10, or 2 to 8, or 2 to 6, or 2 to 4 carbon atoms.

The term “oxo” refers to a ═O functional group.

The term “hydroxyl” refers to a —OH functional group.

The term “cycloalkyl” means a mono- or multi-ring structure that consists of carbon as ring members. A cycloalkyl may be 3 to 16 membered, or 4 to 14 membered, or 5 to 11 membered, or 6 to 10 membered, or 6 to 9 membered, or 6 to 8 membered, or 6 to 7 membered. A multi-ring cycloalkyl may include two to four rings, which may be attached in a pendent manner, fused, or form a bridged system. Illustrative examples of monocyclic cycloalkyls include cyclopropyl, cyclobutyl, cyclopentyl, cyclohexyl, cycloheptyl, and cyclooctyl.

The term “heterocyclyl” means a saturated or unsaturated mono- or multi-ring structure, wherein one or more ring members is a non-carbon atom, e.g., N, S, P, or O. The term “heterocycle” includes all possible isomeric forms of the heterocycles, e.g., pyrrolyl includes 1H-pyrrolyl and 2H-pyrrolyl. In some embodiments, a heterocycle described herein comprises at least one N, S, or O.

The term “aryl” means a fully unsaturated (“aromatic”) mono- or multi-ring structure, which may be 3 to 16 membered, or 4 to 14 membered, or 5 to 11 membered, or 6 to 10 membered, or 6 to 9 membered, or 6 to 8 membered, or 6 to 7 membered. Examples of such moieties include substituted or unsubstituted phenyl (or benzyl). As used herein, a ring “member” refers to an atom as part of the ring structure that is generally attached to at least two other ring members. The term “aryl” refers to an aromatic ring structure containing carbons as ring members.

The term “heteroaryl” refers to an aromatic ring structure comprising carbon and at least one heteroatom, e.g., nitrogen, sulfur, and/or oxygen. As used herein, an “N-heteroaryl,” “S-heteroaryl,” or “O-heteroaryl” refer to a heteroaryl containing at least one nitrogen, sulfur, or oxygen, respectively, as a ring member.

The term “substituted” means that any one or more hydrogen atoms is replaced with any suitable substituent, provided that the normal valency is not exceeded and the replacement results in a stable compound. Suitable substituents include but are not limited to alkyl, alkylaryl, aryl, heteroaryl, halo, hydroxyl, carboxylate, alkoxyl, alkenyl, alkynyl, sulfonyl, amino, cyano, and oxo.

### Glycoside Hydrolases

Polyphenol aglycones are the hydrolysis product of their corresponding phenolic glycosides, which have the general structure of Formula I:

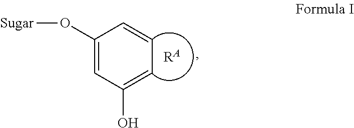

wherein the sugar is a monosaccharide or a disaccharide, and R^{A} is cycloalkyl or heterocyclyl optionally substituted with one or more oxo, alkyl, alkoxyl, alkenyl, alkynyl, hydroxyl, cycloalkyl, heterocyclyl, aryl, or heteroaryl substituents, wherein the cycloalkyl, heterocyclyl, aryl, or heteroaryl is unsubstituted or substituted with one or more oxo, alkyl, alkoxyl, alkenyl, alkynyl, or hydroxyl substituents.

Exemplary polyphenol aglycones include, but are not limited to, naringenin, eriodictyol, and hesperetin. In some embodiments, the polyphenol aglycone is hesperetin. Hesperetin ((2S)-3′,5,7-Trihydroxy-4′-methoxyflavan-4-one) is the aglycone of the phenolic glycoside compound hesperidin, the main flavonoid in citrus fruits such as grapefruits, lemons, and sweet oranges. Hesperetin is used in the food and beverage industry as a debittering agent, and may also have antioxidant, anti-inflammatory, anti-allergic, hypolipidemic, vasoprotective, and anticarcinogenic actions. See, e.g., Wdowiak et al., Nutrients (2022) 14 (13): 2647.

Engineered biosynthesis pathways for hesperetin from compounds such as caffeic acid and naringenin have been described (Liu et al, J Biotechnol (2022) 347:67-76; Hanko et al., BMC Res Notes (2023) 16:343). Enzymatic hydrolysis of hesperidin to produce hesperetin and rutinose has also been described. For example, Weiz et al., Appl Microbiol Biotechnol (2019) 103:9493-9504, report using a glycoside hydrolase from Acremonium sp. DSM 24697 to produce hesperetin from hesperidin in an aqueous solution-based reaction.

The present inventors have discovered alternative glycoside hydrolases that provide improved production of polyphenol aglycones, e.g., hesperetin, from phenolic glycosides, e.g., hesperidin, and that such glycoside hydrolases of the present disclosure comprise a conserved active site. In some embodiments, the glycoside hydrolases of the present disclosure retain high catalytic activity for longer periods of time as compared to prior glycoside hydrolases for hesperetin production. In some embodiments, the glycoside hydrolases of the present disclosure are advantageously capable of solid state catalysis, in which the glycoside hydrolase is solubilized in an aqueous solution, the phenolic glycoside substrate (e.g., hesperidin) is contacted with the solution in solid form, and the polyphenol aglycone product (e.g., hesperetin) is precipitated substantially upon its formation. In some embodiments, a solid state catalysis reaction advantageously allows for simpler reaction conditions, improves reaction flux, and/or results in higher yield of the product.

In some embodiments, the glycoside hydrolases of the present disclosure are advantageously capable of maintaining a substantially high enzyme activity in a solid-state catalysis reaction over an extended period of time at high reaction temperatures, e.g., 52° C., as compared to the glycoside hydrolases described in Weiz et al., Appl Microbiol Biotechnol (2019) 103:9493-9504. In some embodiments, the glycoside hydrolases of the present disclosure are capable of maintaining a substantially high enzyme activity, e.g., at least about 70%, at least about 75%, or at least about 80% conversion from hesperidin to hesperetin, in a solid state catalysis reaction over a period of at least 1, 2, 3, 4, 5, 6, 7, 8, 9, or 10 hours at about 45° C. to about 60° C., or about 48° C. to about 55° C., or about 52° C. In some embodiments, the glycoside hydrolases of the present disclosure are capable of maintaining a substantially high enzyme activity, e.g., about 100% conversion from hesperidin to hesperetin, in a solid state catalysis reaction over a period of at least 1, 2, 3, 4, 5, or 6 hours at about 45° C. to about 60° C., or about 48° C. to about 55° C., or about 52° C. In some embodiments, the glycoside hydrolase of the present disclosure is capable of at least about 10%, at least about 15%, or at least about 20% conversion from hesperidin to hesperetin, in a solid state catalysis reaction at 24 hours following reaction initiation, wherein the reaction is performed at about 45° C. to about 60° C. about 48° C. to about 55° C., or about 52° C.

In some embodiments, the glycoside hydrolases described herein are advantageously capable of converting a phenolic glycoside (e.g., hesperidin) to polyphenol aglycone (e.g., hesperetin) with substantially higher substrate loading conditions, e.g., as compared to the known glycoside hydrolase described in Weiz et al., Appl Microbiol Biotechnol (2019) 103:9493-9504. In some embodiments, the glycoside hydrolases of the present disclosure are capable of converting at least 50 g/L, at least 100 g/L, at least 150 g/L, or at least 200 g/L of hesperidin with at least 80%, at least 85%, or at least 90% conversion rate.

In some embodiments, the present disclosure provides a composition comprising a glycoside hydrolase as described herein, and one or both of a phenolic glycoside (e.g., hesperidin) and its corresponding polyphenol aglycone (e.g., hesperetin). In some embodiments, the composition comprises the glycoside hydrolase and hesperidin. In some embodiments, the composition comprises the glycoside hydrolase and hesperetin. In some embodiments, the composition comprises the glycoside hydrolase, hesperidin, and hesperetin. In some embodiments, the composition comprises the glycoside hydrolase and hesperidin, and the glycoside hydrolase hydrolyzes the hesperidin to form hesperetin, thereby producing a composition comprising the glycoside hydrolase and hesperetin. In some embodiments, the glycoside hydrolase comprises an active site as described below.

In some embodiments, the active site of the glycoside hydrolase comprises about 1 to about 10 regions in the amino acid sequence of the glycoside hydrolase. In some embodiments, the active site comprises about 2 to about 8 regions in the amino acid sequence of the glycoside hydrolase. In some embodiments, the active site comprises about 3 to about 7 regions in the amino acid sequence of the glycoside hydrolase. In some embodiments, the active site comprises about 4 to about 6 regions in the amino acid sequence of the glycoside hydrolase. In some embodiments, the active site comprises about 5 or about 6 regions in the amino acid sequence of the glycoside hydrolase. In some embodiments, the active site comprises at least two regions in the glycoside hydrolase, wherein each of the regions is separated by one or more non-active site residues, and wherein the regions form the active site in the three-dimensional structure of the glycoside hydrolase. In some embodiments, the regions are in proximity to each other in the three-dimensional structure of the glycoside hydrolase.

In some embodiments, the active site of the glycoside hydrolase comprises: a first region comprising at least 70% identity to HQEAII (SEQ ID NO: 1): a second region comprising at least 70% identity to DIHSLPGGLNGMGLGE (SEQ ID NO:2); a third region comprising at least 70% identity to PINEPVDNRDITKFGTP (SEQ ID NO:3): a fourth region comprising at least 70% identity to FRPVDYWAKHFAASTNIVFDVHNYYFAGRPT (SEQ ID NO:4); and a fifth region comprising at least 70% identity to GSAYWTWKFFGNVPVDGEGTQGDYWNY (SEQ ID NO:5). In some embodiments, the active site of the glycoside hydrolases comprises a first region comprising at least 80% identity to SEQ ID NO: 1: a second region comprising at least 80% identity to SEQ ID NO:2: a third region comprising at least 80% identity to SEQ ID NO:3: a fourth region comprising at least 80% identity to SEQ ID NO:4; and a fifth region comprising at least 80% identity to SEQ ID NO:5. In some embodiments, the active site of the glycoside hydrolases comprises a first region comprising at least 90% identity to SEQ ID NO: 1: a second region comprising at least 90% identity to SEQ ID NO: 2: a third region comprising at least 90% identity to SEQ ID NO:3: a fourth region comprising at least 90% identity to SEQ ID NO:4; and a fifth region comprising at least 90% identity to SEQ ID NO: 5. In some embodiments, the active site of the glycoside hydrolases comprises a first region comprising SEQ ID NO: 1: a second region comprising SEQ ID NO:2: a third region SEQ ID NO:3: a fourth region comprising SEQ ID NO:4; and a fifth region comprising SEQ ID NO:5. In some embodiments, the composition of the present disclosure comprises a glycoside hydrolase comprising an active site as described herein; and one or both of hesperidin and hesperetin.

In some embodiments, the first region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 1. In some embodiments, the second region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:2. In some embodiments, the third region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:3. In some embodiments, the fourth region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:4. In some embodiments, the fifth region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:5.

In some embodiments, the first region comprises 0 or 1 mutations as compared to SEQ ID NO: 1. In some embodiments, the second region comprises 0, 1, 2, 3, 4, 5, 6, 7, or 8 mutations as compared to SEQ ID NO:2. In some embodiments, the third region comprises 0, 1, 2, 3, 4, or 5 mutations as compared to SEQ ID NO:3. In some embodiments, the fourth region comprises 0, 1 2, 3, 4, 5, 6, 7, 8, or 9 mutations as compared to SEQ ID NO:4. In some embodiments, the fifth region comprises 0, 1 2, 3, 4, 5, 6, 7, 8, or 9 mutations as compared to SEQ ID NO:5. In some embodiments, each of the first, second, third, fourth, and fifth regions comprise 0 mutations relative to SEQ ID NOs: 1-5, respectively. In some embodiments, the first region comprises 0 or 1 mutations relative to SEQ ID NO: 1; the second region comprises 0 mutations relative to SEQ ID NO:2; the third region comprises 0 or 1 mutations relative to SEQ ID NO:3; the fourth region comprises 0, 1 2, 3, 4, 5, 6, 7, 8, or 9 mutations relative to SEQ ID NO:4; and the fifth region comprises 0, 1, or 2 mutations relative to SEQ ID NO:5. In some embodiments, the glycoside hydrolase active site comprises a combination of the first, second, third, fourth, and fifth regions as shown in Table 1.

TABLE 1
Combinations.

| | 1^{st} region | 2^{nd} region | 3^{rd} region | 4^{th} region | 5^{th} region |
|---|---|---|---|---|---|
| | SEQ ID | SEQ ID | SEQ ID | SEQ ID | SEQ ID |
| Combination | NO: 1 | NO: 2 | NO: 3 | NO: 4 | NO: 5 |
| # | number of mutations in referenced sequence | | | | |
| 1 | 0 | 0 | 0 | 0 | 0 |
| 2 | 1 | 0 | 0 | 0 | 0 |
| 3 | 0 | 0 | 1 | 0 | 0 |
| 4 | 0 | 0 | 0 | 1 | 0 |
| 5 | 0 | 0 | 0 | 2 | 0 |
| 6 | 0 | 0 | 0 | 3 | 0 |
| 7 | 0 | 0 | 0 | 4 | 0 |
| 8 | 0 | 0 | 0 | 5 | 0 |
| 9 | 0 | 0 | 0 | 6 | 0 |
| 10 | 0 | 0 | 0 | 7 | 0 |
| 11 | 0 | 0 | 0 | 8 | 0 |
| 12 | 0 | 0 | 0 | 9 | 0 |
| 13 | 0 | 0 | 0 | 0 | 1 |
| 14 | 0 | 0 | 0 | 0 | 2 |
| 15 | 1 | 0 | 1 | 0 | 0 |
| 16 | 1 | 0 | 0 | 1 | 0 |
| 17 | 1 | 0 | 0 | 2 | 0 |
| 18 | 1 | 0 | 0 | 3 | 0 |
| 19 | 1 | 0 | 0 | 4 | 0 |
| 20 | 1 | 0 | 0 | 5 | 0 |
| 21 | 1 | 0 | 0 | 6 | 0 |
| 22 | 1 | 0 | 0 | 7 | 0 |
| 23 | 1 | 0 | 0 | 8 | 0 |
| 24 | 1 | 0 | 0 | 9 | 0 |
| 25 | 1 | 0 | 0 | 0 | 1 |
| 26 | 1 | 0 | 0 | 0 | 2 |
| 27 | 0 | 0 | 1 | 1 | 0 |
| 28 | 0 | 0 | 1 | 2 | 0 |
| 29 | 0 | 0 | 1 | 3 | 0 |
| 30 | 0 | 0 | 1 | 4 | 0 |
| 31 | 0 | 0 | 1 | 5 | 0 |
| 32 | 0 | 0 | 1 | 6 | 0 |
| 33 | 0 | 0 | 1 | 7 | 0 |
| 34 | 0 | 0 | 1 | 8 | 0 |
| 35 | 0 | 0 | 1 | 9 | 0 |
| 36 | 0 | 0 | 0 | 1 | 1 |
| 37 | 0 | 0 | 0 | 2 | 1 |
| 38 | 0 | 0 | 0 | 3 | 1 |
| 39 | 0 | 0 | 0 | 4 | 1 |
| 40 | 0 | 0 | 0 | 5 | 1 |
| 41 | 0 | 0 | 0 | 6 | 1 |
| 42 | 0 | 0 | 0 | 7 | 1 |
| 43 | 0 | 0 | 0 | 8 | 1 |
| 44 | 0 | 0 | 0 | 9 | 1 |
| 45 | 0 | 0 | 0 | 1 | 2 |
| 46 | 0 | 0 | 0 | 2 | 2 |
| 47 | 0 | 0 | 0 | 3 | 2 |
| 48 | 0 | 0 | 0 | 4 | 2 |
| 49 | 0 | 0 | 0 | 5 | 2 |
| 50 | 0 | 0 | 0 | 6 | 2 |
| 51 | 0 | 0 | 0 | 7 | 2 |
| 52 | 0 | 0 | 0 | 8 | 2 |
| 53 | 0 | 0 | 0 | 9 | 2 |
| 54 | 1 | 0 | 1 | 1 | 0 |
| 55 | 1 | 0 | 1 | 2 | 0 |
| 56 | 1 | 0 | 1 | 3 | 0 |
| 57 | 1 | 0 | 1 | 4 | 0 |
| 58 | 1 | 0 | 1 | 5 | 0 |
| 59 | 1 | 0 | 1 | 6 | 0 |
| 60 | 1 | 0 | 1 | 7 | 0 |
| 61 | 1 | 0 | 1 | 8 | 0 |
| 62 | 1 | 0 | 1 | 9 | 0 |
| 63 | 1 | 0 | 1 | 0 | 1 |
| 64 | 1 | 0 | 1 | 0 | 2 |
| 65 | 1 | 0 | 0 | 1 | 1 |
| 66 | 1 | 0 | 0 | 2 | 1 |
| 67 | 1 | 0 | 0 | 3 | 1 |
| 68 | 1 | 0 | 0 | 4 | 1 |
| 69 | 1 | 0 | 0 | 5 | 1 |
| 70 | 1 | 0 | 0 | 6 | 1 |
| 71 | 1 | 0 | 0 | 7 | 1 |
| 72 | 1 | 0 | 0 | 8 | 1 |
| 73 | 1 | 0 | 0 | 9 | 1 |
| 74 | 1 | 0 | 0 | 1 | 2 |
| 75 | 1 | 0 | 0 | 2 | 2 |
| 76 | 1 | 0 | 0 | 3 | 2 |
| 77 | 1 | 0 | 0 | 4 | 2 |
| 78 | 1 | 0 | 0 | 5 | 2 |
| 79 | 1 | 0 | 0 | 6 | 2 |
| 80 | 1 | 0 | 0 | 7 | 2 |
| 81 | 1 | 0 | 0 | 8 | 2 |
| 82 | 1 | 0 | 0 | 9 | 2 |
| 83 | 0 | 0 | 1 | 1 | 1 |
| 84 | 0 | 0 | 1 | 2 | 1 |
| 85 | 0 | 0 | 1 | 3 | 1 |
| 86 | 0 | 0 | 1 | 4 | 1 |
| 87 | 0 | 0 | 1 | 5 | 1 |
| 88 | 0 | 0 | 1 | 6 | 1 |
| 89 | 0 | 0 | 1 | 7 | 1 |
| 90 | 0 | 0 | 1 | 8 | 1 |
| 91 | 0 | 0 | 1 | 9 | 1 |
| 92 | 0 | 0 | 1 | 1 | 2 |
| 93 | 0 | 0 | 1 | 2 | 2 |
| 94 | 0 | 0 | 1 | 3 | 2 |
| 95 | 0 | 0 | 1 | 4 | 2 |
| 96 | 0 | 0 | 1 | 5 | 2 |
| 97 | 0 | 0 | 1 | 6 | 2 |
| 98 | 0 | 0 | 1 | 7 | 2 |
| 99 | 0 | 0 | 1 | 8 | 2 |
| 100 | 0 | 0 | 1 | 9 | 2 |
| 101 | 1 | 0 | 1 | 1 | 1 |
| 102 | 1 | 0 | 1 | 2 | 1 |
| 103 | 1 | 0 | 1 | 3 | 1 |
| 104 | 1 | 0 | 1 | 4 | 1 |
| 105 | 1 | 0 | 1 | 5 | 1 |
| 106 | 1 | 0 | 1 | 6 | 1 |
| 107 | 1 | 0 | 1 | 7 | 1 |
| 108 | 1 | 0 | 1 | 8 | 1 |
| 109 | 1 | 0 | 1 | 9 | 1 |
| 110 | 1 | 0 | 1 | 1 | 2 |
| 111 | 1 | 0 | 1 | 2 | 2 |
| 112 | 1 | 0 | 1 | 3 | 2 |
| 113 | 1 | 0 | 1 | 4 | 2 |
| 114 | 1 | 0 | 1 | 5 | 2 |
| 115 | 1 | 0 | 1 | 6 | 2 |
| 116 | 1 | 0 | 1 | 7 | 2 |
| 117 | 1 | 0 | 1 | 8 | 2 |
| 118 | 1 | 0 | 1 | 9 | 2 |

In some embodiments, the first region is N-terminal to the second region: the second region is N-terminal to the third region: the third region is N-terminal to the fourth region; and the fourth region is N-terminal to the fifth region in the glycoside hydrolase. In some embodiments, the glycoside hydrolase comprises about 1 to 25, about 1 to 24, about 1 to 23, about 1 to 22, about 1 to 21, about 1 to 20, about 1 to 18, about 1 to 15, about 1 to 12, or about 1 to 10 amino acids between the N-terminus and the first region. In some embodiments, the glycoside hydrolase comprises about 1 to 100, about 1 to 99, about 1 to 98, about 1 to 97, about 1 to 96, about 1 to 95, about 1 to 94, about 1 to 93, about 1 to 92, about 1 to 91, about 1 to 90, about 1 to 85, about 1 to 80, about 1 to 75, or about 1 to 70 amino acids between the first region and the second region. In some embodiments, the glycoside hydrolase comprises about 1 to 45, about 1 to 44, about 1 to 43, about 1 to 42, about 1 to 41, about 1 to 40, about 1 to 39, about 1 to 38, about 1 to 37, about 1 to 36, about 1 to 35, about 1 to 30, about 1 to 25, or about 1 to 20 amino acids between the second region and the third region. In some embodiments, the glycoside hydrolase comprises about 1 to 40, about 1 to 39, about 1 to 38, about 1 to 37, about 1 to 36, about 1 to 35, about 1 to 34, about 1 to 33, about 1 to 32, about 1 to 31, about 1 to 30, about 1 to 25, about 1 to 20, or about 1 to 15 amino acids between the third region and the fourth region. In some embodiments, the glycoside hydrolase comprises about 1 to 65, about 1 to 64, about 1 to 63, about 1 to 62, about 1 to 61, about 1 to 60, about 1 to 59, about 1 to 58, about 1 to 57, about 1 to 56, about 1 to 55, about 1 to 50, about 1 to 45, or about 1 to 40 amino acids between the fourth region and the fifth region. In some embodiments, the glycoside hydrolase comprises about 1 to 20, about 1 to 19, about 1 to 18, about 1 to 17, about 1 to 16, about 1 to 15, about 1 to 12, about 1 to 10, or about 1 to 5 amino acids between the fifth region and the C-terminus. In some embodiments, the glycoside hydrolase comprises, in the following order: the first region, the second region, the third region, the fourth region, and the fifth region. As described herein, the number of amino acid “between” the indicated regions refers to the number of acids from the last residue of the first indicated region to the first residue of the second indicated region.

In some embodiments, the active site of the glycoside hydrolase further comprises a sixth region. In some embodiments, the sixth region is located between the fourth region and the fifth region described herein. In some embodiments, the glycoside hydrolase comprises about 1 to 25, about 1 to 24, about 1 to 23, about 1 to 22, about 1 to 20, about 1 to 19, about 1 to 18, about 1 to 17, about 1 to 16, about 1 to 15, about 1 to 13, about 1 to 10, about 1 to 8, or about 1 to 5 amino acids between the fourth region and the sixth region. In some embodiments, the glycoside hydrolase comprises, in the following order: the first region, the second region, the third region, the fourth region, the sixth region, and the fifth region.

In some embodiments, the sixth region comprises at least 70% identity to KFPVFVGEWSIQAA (SEQ ID NO:6). In some embodiments, the sixth region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:6. In some embodiments, the sixth region comprises at least 70% identity to CSDAKXXXXXXXXKFPVFVGEWSIQAA (SEQ ID NO:7), wherein X is any amino acid. In some embodiments, the sixth region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:7. In some embodiments, the sixth region comprises at least 70% identity to CSDAKDIVSTASPKFPVFVGEWSIQAA (SEQ ID NO:8). In some embodiments, the sixth region comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:8. In some embodiments, the glycoside hydrolase active site comprises a combination according to Table 1, wherein the combination further comprises a sixth region comprising 0 or 1 mutations as compared to SEQ ID NO:6. In some embodiments, the glycoside hydrolase active site comprises a combination according to Table 1, wherein the combination further comprises a sixth region comprising 0, 1, or 2 mutations as compared to SEQ ID NO:7.

In some embodiments, the active site of the glycoside hydrolase comprises: a first region comprising at least 70%, at least 80%, at least 85%, at least 90%, at least 95%, or at least 100% sequence identity to SEQ ID NO: 1: a second region comprising at least 70%, at least 80%, at least 85%, at least 90%, at least 95%, or at least 100% sequence identity to SEQ ID NO:2: a third region comprising at least 70%, at least 80%, at least 85%, at least 90%, at least 95%, or at least 100% sequence identity to SEQ ID NO:3: a fourth region comprising at least 70%, at least 80%, at least 85%, at least 90%, at least 95%, or at least 100% sequence identity to SEQ ID NO:4: a sixth region comprising at least 70%, at least 80%, at least 85%, at least 90%, at least 95%, or at least 100% sequence identity to SEQ ID NO:6; and a fifth region comprising at least 70%, at least 80%, at least 85%, at least 90%, at least 95%, or at least 100% sequence identity to SEQ ID NO:5. In some embodiments, the composition of the present disclosure comprises a glycoside hydrolase comprising an active site as described herein; and one or both of hesperidin and hesperetin.

In some embodiments, the active site of the glycoside hydrolase comprises: a first region comprising at least 70%, at least 80%, at least 85%, at least 90%, at least 95%, or at least 100% sequence identity to SEQ ID NO: 1: a second region comprising at least 70%, at least 80%, at least 85%, at least 90%, at least 95%, or at least 100% sequence identity to SEQ ID NO:2: a third region comprising at least 70%, at least 80%, at least 85%, at least 90%, at least 95%, or at least 100% sequence identity to SEQ ID NO:3: a fourth region comprising at least 70%, at least 80%, at least 85%, at least 90%, at least 95%, or at least 100% sequence identity to SEQ ID NO:4: a sixth region comprising at least 70%, at least 80%, at least 85%, at least 90%, at least 95%, or at least 100% sequence identity to SEQ ID NO:7; and a fifth region comprising at least 70%, at least 80%, at least 85%, at least 90%, at least 95%, or at least 100% sequence identity to SEQ ID NO:5. In some embodiments, the composition of the present disclosure comprises a glycoside hydrolase comprising an active site as described herein; and one or both of hesperidin and hesperetin.

In some embodiments, the active site of the glycoside hydrolase comprises: a first region comprising at least 70%, at least 80%, at least 85%, at least 90%, at least 95%, or at least 100% sequence identity to SEQ ID NO: 1: a second region comprising at least 70%, at least 80%, at least 85%, at least 90%, at least 95%, or at least 100% sequence identity to SEQ ID NO:2: a third region comprising at least 70%, at least 80%, at least 85%, at least 90%, at least 95%, or at least 100% sequence identity to SEQ ID NO:3: a fourth region comprising at least 70%, at least 80%, at least 85%, at least 90%, at least 95%, or at least 100% sequence identity to SEQ ID NO:4: a sixth region comprising at least 70%, at least 80%, at least 85%, at least 90%, at least 95%, or at least 100% sequence identity to SEQ ID NO:8; and a fifth region comprising at least 70%, at least 80%, at least 85%, at least 90%, at least 95%, or at least 100% sequence identity to SEQ ID NO:5. In some embodiments, the composition of the present disclosure comprises a glycoside hydrolase comprising an active site as described herein; and one or both of hesperidin and hesperetin.

In some embodiments, the glycoside hydrolases of the present disclosure are derived from Fusarium oxysporum, Fusarium pseudograminearum, Fusarium tricinctum, or Gibberella moniliformis (also known as Fusarium verticillioides). In some embodiments, the glycoside hydrolase of the present disclosure comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 9-12. In some embodiments, the present disclosure provides a composition comprising (a) the glycoside hydrolase comprising at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 9-12; and (b) one or both of hesperidin and hesperetin.

SEQ ID NO:9 describes the amino acid sequence of a glucan 1,3-beta-glucosidase from Fusarium oxysporum. Fusarium oxysporum is a fungus that infects non-citrus crops such as tomatoes, bananas, cotton, peppers, and cucumbers, none of which are known to contain hesperidin or hesperetin. SEQ ID NO: 10 describes the amino acid sequence of a glucan 1,3-beta-glucosidase from Fusarium pseudograminearum. Fusarium pseudograminearum is a fungus that infects grasses and cereal crops, particularly wheat and barley, and causes crown rot. Fusarium pseudograminearum is not found in citrus plants. SEQ ID NO: 11 describes the amino acid sequence of a glucan 1,3-beta-glucosidase from Fusarium tricinctum. Fusarium tricinctum is a fungus that is primarily associated with grains such as wheat, barley, and oats, as well as non-citrus crops, and is known to produce mycotoxins. Fusarium tricinctum does not infect citrus plants. SEQ ID NO: 12 describes the amino acid sequence of a glucan 1,3-beta-glucosidase from Gibberella moniliformis. Gibberella moniliformis is a fungus that is primarily found in maize plants and is associated with stalk rot and car rot in corn. Gibberella moniliformis does not infect citrus plants. As none of these fungal species are associated with any naturally occurring plants that may contain hesperidin and/or hesperetin, the glycoside hydrolases derived from these fungal species are also not found in nature with hesperidin or hesperetin. Accordingly, the compositions provided herein, which comprise the glycoside hydrolases described herein and one or both of hesperidin and hesperetin, are non-naturally occurring compositions.

In some embodiments, the glycoside hydrolase comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to SEQ ID NO:9. In some embodiments, the glycoside hydrolase comprises SEQ ID NO:9. In some embodiments, the glycoside hydrolase is capable of producing hesperetin, e.g., from hesperidin as described herein. In some embodiments, the composition of the present disclosure comprises the glycoside hydrolase; and one or both of hesperidin and hesperetin.

In some embodiments, the glycoside hydrolase comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to SEQ ID NO: 10. In some embodiments, the glycoside hydrolase comprises SEQ ID NO:10. In some embodiments, the glycoside hydrolase is capable of producing hesperetin, e.g., from hesperidin as described herein. In some embodiments, the composition of the present disclosure comprises the glycoside hydrolase; and one or both of hesperidin and hesperetin.

In some embodiments, the glycoside hydrolase comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to SEQ ID NO:11. In some embodiments, the glycoside hydrolase comprises SEQ ID NO: 10. In some embodiments, the glycoside hydrolase is capable of producing hesperetin, e.g., from hesperidin as described herein. In some embodiments, the composition of the present disclosure comprises the glycoside hydrolase; and one or both of hesperidin and hesperetin.

In some embodiments, the glycoside hydrolase comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to SEQ ID NO: 12. In some embodiments, the glycoside hydrolase comprises SEQ ID NO: 12. In some embodiments, the glycoside hydrolase is capable of producing hesperetin, e.g., from hesperidin as described herein. In some embodiments, the composition of the present disclosure comprises the glycoside hydrolase; and one or both of hesperidin and hesperetin.

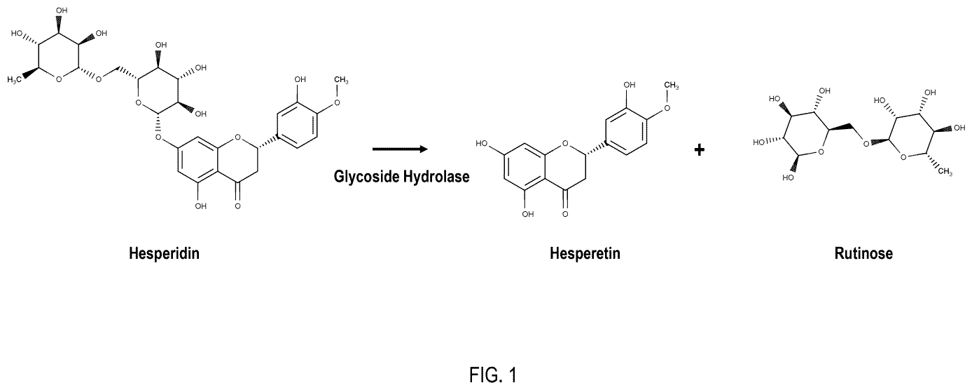

In some embodiments, the composition comprises the glycoside hydrolase and hesperidin. In some embodiments, the glycoside hydrolase hydrolyzes the hesperidin to produce hesperetin. The hydrolysis of hesperidin to produce hesperetin and rutinose is illustrated in FIG. 1. In some embodiments, the composition comprises the glycoside hydrolase and hesperetin. In some embodiments, the hesperetin is produced from hydrolysis of the hesperidin as described herein. In some embodiments, the glycoside hydrolase is solubilized in an aqueous solution, and the hesperidin and/or hesperetin are in solid form. In some embodiments, the aqueous solution is a buffer solution for the glycoside hydrolase. In some embodiments, the aqueous solution is a buffer at a pH of about 3.5 to about 7, or about 3.7 to about 6.5, or about 4 to about 6, or about 4.5 to about 5.8, or about 5 to about 5.6, or about 5.2 to about 5.5, or about 5.3 to about 5.4.

In some embodiments, the composition further comprises an organic solvent, a surfactant, or both. In some embodiments, the composition comprises the glycoside hydrolase, one or both of hesperidin and hesperetin, and the organic solvent. Exemplary organic solvents include, but are not limited to, dimethyl sulfoxide (DMSO), dimethylformamide (DMF), gamma-butyrolactone (GBL), N-methyl-pyrrolidone (NMP), dimethylacetamide (DMAc), chloroform, toluene, chlorobenzene, acetone, and alcohols such as methanol, ethanol, and isopropyl alcohol. In some embodiments, the organic solvent is DMSO. In some embodiments, the composition comprises the glycoside hydrolase, one or both of hesperidin and hesperetin, and DMSO.

In some embodiments, the composition comprises the glycoside hydrolase, one or both of hesperidin and hesperetin, and the surfactant. In some embodiments, the surfactant comprises an ionic surfactant, e.g., sulfates, quaternary ammonium compounds, and betaines. In some embodiments, the surfactant comprises a nonionic surfactant, e.g., polysorbates, sorbitans and sorbitan esters, PEG-based surfactants, and polyethylene oxide surfactants. In some embodiments, the surfactant comprises 2-[4-(2,4,4-trimethylpentan-2-yl)phenoxy]ethanol, also known as TRITON™ X-100. In some embodiments, the surfactant comprises a polysorbate surfactant, also known as a TWEEN® surfactant. Non-limiting examples of TWEEN® surfactants include polyoxyethylene (20) sorbitan monolaurate (polysorbate 20 or TWEEN® 20), polyoxyethylene (20) sorbitan monopalmitate (polysorbate 40 or TWEEN® 40), polyoxyethylene (20) sorbitan monostearate (polysorbate 60 or TWEEN® 60), and polyoxyethylene (20) sorbitan monooleate (polysorbate 80 or TWEEN® 80). In some embodiments, the surfactant comprises TRITON® X-100, TWEEN® or a combination thereof. In some embodiments, the surfactant comprises TRITON™ X-100, TWEEN® 20, TWEEN® 80, or a combination thereof. In some embodiments, the composition comprises the glycoside hydrolase, one or both of hesperidin and hesperetin, and TRITON™ X-100. In some embodiments, the composition comprises the glycoside hydrolase, one or both of hesperidin and hesperetin, and TWEEN®.

### Nucleic Acids

In some embodiments, the present disclosure provides a nucleic acid encoding a glycoside hydrolase described herein. In some embodiments, the nucleic acid is operably linked to a heterologous regulatory element. As described herein, the “heterologous” regulatory element is a regulatory element that is not naturally found to be associated or operably linked with the referenced nucleic acid. In some embodiments, the heterologous regulatory element comprises a promoter, an enhancer, a silencer, a response element, or a combination thereof. In some embodiments, the heterologous regulatory element is a promoter. In some embodiments, the promoter is a constitutive promoter. In some embodiments, the promoter is an inducible promoter. In some embodiments, the inducible promoter is induced in the presence of an agent such as methanol, glucose, or galactose, or upon an environmental change such as a change to anaerobic conditions. In some embodiments, the heterologous regulatory element is a fungal regulatory element. In some embodiments, the heterologous regulatory element is a promoter for expression in Saccharomyces cerevisiae, Pichia pastoris, and/or Trichoderma reesei. Non-limiting examples of fungal regulatory elements include promoters such as TEF1, TDH3, PGK1, TPI1, CCW12, ENO2, GAL1/2/7/10, AOX1, GAP, cDNA1, ENO1, GPD1, PDC1, PKI1, RP2, and CBH1. Further examples of promoters and other regulatory elements can be found, e.g., using the PRODORIC2 database.

In some embodiments, the present disclosure provides a nucleic acid operably linked to a heterologous regulatory element, wherein the nucleic acid encodes a glycoside hydrolase comprising a first region comprising at least 70%, at least 80%, at least 85%, at least 90%, at least 95%, or at least 100% sequence identity to SEQ ID NO:1: a second region comprising at least 70%, at least 80%, at least 85%, at least 90%, at least 95%, or at least 100% sequence identity to SEQ ID NO:2: a third region comprising at least 70%, at least 80%, at least 85%, at least 90%, at least 95%, or at least 100% sequence identity to SEQ ID NO:3: a fourth region comprising at least 70%, at least 80%, at least 85%, at least 90%, at least 95%, or at least 100% sequence identity to SEQ ID NO:4; and a fifth region comprising at least 70%, at least 80%, at least 85%, at least 90%, at least 95%, or at least 100% sequence identity to SEQ ID NO:5. In some embodiments, the active site of the glycoside hydrolase comprises a combination of the first, second, third, fourth, and fifth regions as shown in Table 1. In some embodiments, the active site of the glycoside hydrolase further comprises a sixth region comprising at least 70%, at least 80%, at least 85%, at least 90%, at least 95%, or at least 100% sequence identity to any one of SEQ ID NOs: 6-8. In some embodiments, operably linked to a heterologous regulatory element, wherein the nucleic acid encodes a glycoside hydrolase comprising at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 9-12. In some embodiments, the active site of the glycoside hydrolase comprises a combination of the first, second, third, fourth, and fifth regions as shown in Table 1, and further comprising a sixth region comprising at least 70%, at least 80%, at least 85%, at least 90%, at least 95%, or at least 100% sequence identity to any one of SEQ ID NOs: 6-8.

In some embodiments, the nucleic acid is linked to a sequence encoding a signal sequence. In some embodiments, the signal sequence directs secretion of a protein upon expression. In some embodiments, the signal sequence is an alpha-factor signal sequence, also referred to as “alpha-factor.” In some embodiments, the nucleic acid is linked at its 5′-end to a sequence encoding an alpha-factor signal sequence. In some embodiments, upon expressing the nucleic acid, the alpha-factor signal sequence is fused to the N-terminus of the glycoside hydrolase. In some embodiments, the alpha-factor comprises at least 90%, at least 91%, at least 92%, at least 93%, at least 94%, at least 95%, at least 96%, at last 97%, at least 98%, at least 99%, or 100% sequence identity to SEQ ID NO: 13.

In some embodiments, the present disclosure provides a vector comprising the nucleic acid encoding the glycoside hydrolase as described herein. In some embodiments, the vector is an expression vector. In some embodiments, the vector is a bacterial expression vector. In some embodiments, the vector is a fungal expression vector. In some embodiments, the vector is an E. coli, Corynebacterium, Bacillus, Ralstonia, Zymomonas, Staphylococcus, Pichia (e.g., Pichia pastoris), Trichoderma (e.g., Trichoderma reesei), Saccharomyces, Candida (e.g., Candida albicans), Yarrowia, or Aspergillus expression vector. In some embodiments, the vector is a Pichia expression vector. Exemplary Pichia expression vectors include, but are not limited to, pPIC9K, pPICZα, pHIL-S1, pGAPZα, pJL-SX, and pBLHIS-SX. In some embodiments, the vector is a Trichoderma expression vector. Exemplary Trichoderma expression vectors include, but are not limited to, pTrEno, pTYGS, pWEF31, and pWEF32. In some embodiments, the vector comprises the sequence encoding the alpha-factor signal sequence described herein.

In some embodiments, the nucleic acid encoding the glycoside hydrolase, and/or the vector comprising the nucleic acid, are introduced into a host cell, as a heterologous nucleic acid. Methods of introducing heterologous nucleic acids and/or vectors into host cells are known to one of ordinary skill in the art. Non-limiting exemplary methods for introducing nucleic acids and/or vectors into host cells include electroporation, conjugation, transduction, natural transformation, calcium phosphate precipitation, DEAE-dextran mediated transfection, liposome-mediated transfection, and particle bombardment. The nucleic acid or vector typically further includes a selectable marker, e.g., neomycin resistance, ampicillin resistance, tetracycline resistance, chloramphenicol resistance, kanamycin resistance, hygromycin resistance, G418 resistance, bleomycin resistance, zeocin resistance, and the like. In some embodiments, the heterologous nucleic acid is present on a plasmid in the host cell. In some embodiments, the nucleic acid is integrated into a genome of the host cell. In some embodiments, the integration is a stable integration. In some embodiments, the nucleic acid is integrated into the host cell genome using a CRISPR system, e.g., CRISPR/Cas9.

In some embodiments, the present disclosure provides an expression system comprising a host cell comprising a heterologous nucleic acid encoding a glycosidic hydrolase as described herein. In some embodiments, the heterologous nucleic acid further comprises a regulatory element for expressing the glycoside hydrolase. In some embodiments, the present disclosure provides a host cell capable of expressing a heterologous glycoside hydrolase described herein. In some embodiments, the host cell is further capable of secreting the glycoside hydrolase after expression. In some embodiments, the glycoside hydrolase is secreted from the host cell due to the presence of a signal sequence described herein, e.g., an alpha-factor signal sequence. In some embodiments, the alpha-factor signal sequence is cleaved upon secretion of the glycoside hydrolase. In some embodiments, the glycoside hydrolase is capable of hydrolyzing a phenolic glycoside to form a polyphenol aglycone. In some embodiments, the phenolic glycoside is hesperidin, and the polyphenol aglycone is hesperetin.

A variety of microorganisms may be suitable as the host cell described herein, so long as the glycoside hydrolase encoded by the nucleic acid and/or to be expressed by the host cell is not native to the host cell (i.e., the glycoside hydrolase is a “heterologous” glycoside hydrolase). For example, in embodiments where the glycoside hydrolase comprises SEQ ID NO:9, the host cell is not Fusarium oxysporum. In embodiments where the glycoside hydrolase comprises SEQ ID NO: 10, the host cell is not Fusarium pseudograminearum. In embodiments where the glycoside hydrolase comprises SEQ ID NO: 11, the host cell is not Fusarium tricinctum. In embodiments where the glycoside hydrolase comprises SEQ ID NO: 12, the host cell is not Gibberella moniliformis. In some embodiments, the host cell is a prokaryotic cell. In some embodiments, the host cell is a eukaryotic cell. In some embodiments, the host cell is capable of expressing and secreting the glycoside hydrolase described herein. In some embodiments, the host cell is bacteria. In some embodiments, the bacteria is Escherichia, Corynebacterium, Pseudomonas. Bacillus, Ralstonia, Zymomonas, Staphylococcus, Clostridium, Salmonella, Rhodococcus, Enterococcus, Alcaligenes, Klebsiella, Paenibacillus, Arthrobacter, Brevibacterium, Lactobacillus, or Lactococcus. In some embodiments, the bacteria is Escherichia, Corynebacterium, Bacillus, Ralstonia, Zymomonas, or Staphylococcus. In some embodiments, the bacteria is Escherichia coli, Corynebacterium glutamicum, Pseudomonas aeruginosa, Bacillus subtilis, Ralstonia eutropha, Zymomonas mobilis, or Staphylococcus aureus. In some embodiments, the host cell is fungi. In some embodiments, the fungi is Saccharomyces, Pichia, Trichoderma, Yarrowia, Aspergillus, or Candida. In some embodiments, the fungi is Saccharomyces cerevisiae, Pichia pastoris, Trichoderma reesei, Yarrowia lipolytica, Aspergillus fumigatus, or Candida albicans. In some embodiments, the host cell is E. coli. In some embodiments, the host cell is Pichia pastoris. In some embodiments, the host cell is Trichoderma reesei.

Further exemplary host cells include, but are not limited to, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile. Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas aeruginosa, Pseudomonas putida, Pseudomonas stutzeri, Pseudomonas fluorescens, Rhodobacter spaeroides, Thermoanaerobacter brockii, Metallosphaera sedula, Leuconostoc mesenteroides, Chloroflexus aurantiacus, Roseiflexus castenholzii, Simmondsia chinensis, Acinetobacter calcoaceticus, Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Caenorhabditis elegans, Corynebacterium glutamicum, Acidaminococcus fermentans, Lactococcus lactis, Lactobacillus plantarum, Streptococcus thermophilus, Enterobacter aerogenes, Saccharomyces cerevisiae, Pichia pastoris, Trichoderma reesei, Yarrowia lipolytica, Aspergillus fumigatus, Candida albicans, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Haloferax mediterranei, Agrobacterium tumefaciens, Achromobacter denitrificans, Fusobacterium nucleatum, Streptomyces clavuligenus, Acinetobacter baumanii, Lachancea kluyveri, Trichomonas vaginalis, Trypanosoma brucei, Pseudomonas stutzeri, Bradyrhizobium japonicum, Mesorhizobium loti, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis, Mycobacterium avium, Mycobacterium marinum, and Tsukamurella paurometabola.

### Methods

In some embodiments, the glycoside hydrolase described herein is capable of hydrolyzing other phenolic glycosides in addition to hesperidin, to produce other polyphenol aglycones in addition to hesperetin. In some embodiments, the present disclosure provides a method of producing a polyphenol aglycone, comprising contacting a phenolic glycoside with a glycoside hydrolase described herein. In some embodiments, the phenolic glycoside is hesperidin, and the polyphenol aglycone is hesperetin. In some embodiments, the glycoside hydrolase comprises an active site comprising: a first region comprising at least 70%, at least 80%, at least 85%, at least 90%, at least 95%, or at least 100% sequence identity to SEQ ID NO: 1: a second region comprising at least 70%, at least 80%, at least 85%, at least 90%, at least 95%, or at least 100% sequence identity to SEQ ID NO: 2: a third region comprising at least 70%, at least 80%, at least 85%, at least 90%, at least 95%, or at least 100% sequence identity to SEQ ID NO:3: a fourth region comprising at least 70%, at least 80%, at least 85%, at least 90%, at least 95%, or at least 100% sequence identity to SEQ ID NO:4; and a fifth region comprising at least 70%, at least 80%, at least 85%, at least 90%, at least 95%, or at least 100% sequence identity to SEQ ID NO:5. In some embodiments, the active site of the glycoside hydrolase comprises a combination of the first, second, third, fourth, and fifth regions as shown in Table 1. In some embodiments, the active site of the glycoside hydrolase further comprises a sixth region comprising at least 70%, at least 80%, at least 85%, at least 90%, at least 95%, or at least 100% sequence identity to any one of SEQ ID NOs: 6-8. In some embodiments, the glycoside hydrolase comprises at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 9-12. In some embodiments, the active site of the glycoside hydrolase comprises a combination of the first, second, third, fourth, and fifth regions as shown in Table 1, and further comprising a sixth region comprising at least 70%, at least 80%, at least 85%, at least 90%, at least 95%, or at least 100% sequence identity to any one of SEQ ID NOs: 6-8.

In some embodiments, the polyphenol aglycone comprises a structure of Formula I:

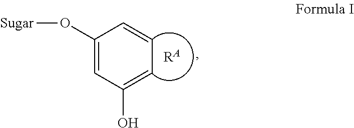

wherein the sugar is a monosaccharide or a disaccharide, and R^{A} is cycloalkyl or heterocyclyl optionally comprising one or more oxo, alkyl, alkoxyl, alkenyl, alkynyl, hydroxyl, cycloalkyl, heterocyclyl, aryl, or heteroaryl substituents, wherein the cycloalkyl, heterocyclyl, aryl, or heteroaryl optionally comprises one or more oxo, alkyl, alkoxyl, alkenyl, alkynyl, or hydroxyl substituents.

In some embodiments, the sugar of the polyphenol aglycone is a monosaccharide. In some embodiments, the monosaccharide is glucose, galactose, glucuronic acid, galactouronic acid, ribose, fructose, apiose, arabinose, xylose, or rhamnose. In some embodiments, the sugar of the polyphenol aglycone is a disaccharide. In some embodiments, the disaccharide comprises two of the monosaccharides described above. In some embodiments, the disaccharide is rutinose, sambubiose, gentiobiose, sophorose, or neohesperidose.

In some embodiments, the R^{A} of the polyphenol aglycone is a cycloalkyl. In some embodiments, the R^{A} of the polyphenol aglycone is a heterocyclyl. In some embodiments, the heterocyclyl comprises one or more heteroatoms independently selected from N, O, and S. In some embodiments, the heterocyclyl comprises O. In some embodiments, the heterocyclyl comprises a saturated bond between two the ring members. In some embodiments, the R^{A} and ring to which it is attached together form a 9-membered bicyclic structure. In some embodiments, the R^{A} and ring to which it is attached together form a 10-membered bicyclic structure. In some embodiments, the R^{A} comprises one or more oxo, alkyl, alkoxyl, alkenyl, alkynyl, hydroxyl, cycloalkyl, heterocyclyl, aryl, or heteroaryl substituents. In some embodiments, the R^{A} comprises more than one substituents, wherein each substituent is at a different position in the R^{A} ring. In some embodiments, the R^{A} comprises more than one substituents, wherein more than one substituents are at the same position in the R^{A} ring. In some embodiments, the R^{A} comprises one, two, three, or four substituents. In some embodiments, the R^{A} comprises an oxo substituent. In some embodiments, the R^{A} comprises an aryl substituent. In some embodiments, the R^{A} comprises an oxo substituent and an aryl substituent, wherein the substituents are at different positions in the R^{A} ring. In some embodiments, the aryl comprises one or more oxo, alkyl, alkoxyl, alkenyl, alkynyl, or hydroxyl substituents. In some embodiments, the aryl comprises a hydroxyl substituent. In some embodiments, the aryl comprises an alkoxyl substituent. In some embodiments, the alkoxyl substituent is a C₁₋₈ alkoxyl, or a C₁₋₆ alkoxyl, or a C₁₋₄ alkoxyl, or a C₁₋₃ alkoxyl, or a C₁₋₂ alkoxyl. In some embodiments, the alkoxyl substituent is a C₁, C₂, C₃, C₄, C₅, C₆, C₇, or C₈ alkoxyl. In some embodiments, the alkoxyl substituent is a C₁ alkoxyl.

In some embodiments, the polyphenol aglycone of the present disclosure comprises a structure of Formula IA:

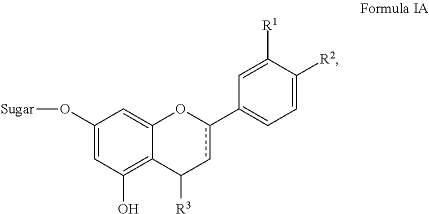

wherein the sugar is a monosaccharide or a disaccharide; R¹ is H, oxo, alkyl, alkoxyl, alkenyl, alkynyl, or hydroxyl; R² is oxo, alkyl, alkoxyl, alkenyl, alkynyl, or hydroxyl; and R³ is oxo, alkyl, alkoxyl, alkenyl, alkynyl, or hydroxyl. In some embodiments, the dotted line represents a single bond. In some embodiments, the dotted line represents a double bond. In some embodiments, R¹ is H. In some embodiments, R¹ is alkoxyl. In some embodiments, R¹ is a C₁₋₈ alkoxyl, or a C₁₋₆ alkoxyl, or a C₁₋₄ alkoxyl, or a C₁₋₃ alkoxyl, or a C₁₋₂ alkoxyl. In some embodiments, the alkoxyl substituent is a C₁, C₂, C₃, C₄, C₅, C₆, C₇, or C₈ alkoxyl. In some embodiments, the alkoxyl substituent is methoxyl. In some embodiments, R¹ is hydroxyl. In some embodiments, R² is hydroxyl. In some embodiments, R³ is oxo. In some embodiments, the dotted line is a single bond, and R¹ is H, R² is hydroxyl, and R³ is oxo. In some embodiments, the dotted line is a double bond, and R¹ is H, R² is hydroxyl, and R³ is oxo. In some embodiments, the dotted line is a single bond, and R¹ is hydroxyl, R² is hydroxyl, and R³ is oxo. In some embodiments, the dotted line is a single bond, and R¹ is alkoxyl (e.g., methoxyl), R² is hydroxyl, and R³ is oxo.

In some embodiments, the polyphenol aglycone of the present disclosure comprises a structure of Formula IB:

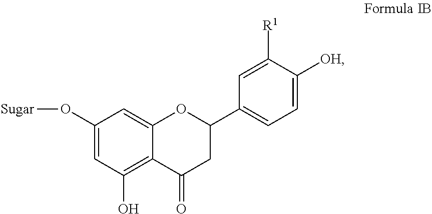

wherein the sugar is a monosaccharide or a disaccharide; and R¹ is H, oxo, alkyl, alkoxyl, alkenyl, alkynyl, or hydroxyl. In some embodiments, R¹ is H. In some embodiments, R¹ is alkoxyl. In some embodiments, R¹ is a C₁₋₈ alkoxyl, or a C₁₋₆ alkoxyl, or a C₁₋₄ alkoxyl, or a C₁₋₃ alkoxyl, or a C₁₋₂ alkoxyl. In some embodiments, the alkoxyl substituent is a C₁, C₂, C₃, C₄, C₅, C₆, C₇, or C₈ alkoxyl. In some embodiments, the alkoxyl substituent is methoxyl. In some embodiments, R¹ is hydroxyl.

In some embodiments, the phenolic glycoside is naringin, and the polyphenol aglycone is naringenin. In some embodiments, the phenolic glycoside is eriocitrin, and the polyphenol aglycone is eriodictyol. In some embodiments, the phenolic glycoside is apiin, apigetrin, vitexin, isovitexin, rhoifolin, or schaftoside, and the polyphenol aglycone is apigenin. In some embodiments, the phenolic glycoside is hesperidin, and the polyphenol aglycone is hesperetin.

As discussed herein, it was discovered that the glycoside hydrolases of the present disclosure are advantageously capable of solid-state enzyme catalysis, thereby providing simpler reaction conditions, improved reaction flux, and higher product yields. In some embodiments, the method of the present disclosure comprises contacting the glycoside hydrolysis solubilized in an aqueous solution, with the phenolic glycoside in solid form. In some embodiments, the polyphenol aglycone produced from the method is also in solid form. In some embodiments, the polyphenol aglycone produced by the method precipitates from the aqueous solution upon formation. In some embodiments, precipitation of the polyphenol aglycone reduces product inhibition of the glycoside hydrolase. In some embodiments, reducing product inhibition increases reaction flux. In some embodiments, reducing product inhibition increases product yield. In some embodiments, the phenolic glycoside is hesperidin, and the polyphenol aglycone is hesperetin.

In some embodiments, the method of the present disclosure comprises contacting the glycoside hydrolase with the phenolic glycoside in the presence of an organic solvent, a surfactant, or both. In some embodiments, the contacting is in the presence of an organic solvent. In some embodiments, the contacting is in the presence of a surfactant. In some embodiments, the contacting is in the presence of an organic solvent and a surfactant. Exemplary organic solvents include, but are not limited to, dimethyl sulfoxide (DMSO), dimethylformamide (DMF), gamma-butyrolactone (GBL). N-methyl-pyrrolidone (NMP), dimethylacetamide (DMAc), acetone, and alcohols such as methanol, ethanol, and isopropyl alcohol. In some embodiments, the organic solvent is DMSO. In some embodiments, the surfactant comprises a nonionic surfactant. e.g., polysorbates, sorbitans. PEG-based surfactants, and polyethylene oxide surfactants. In some embodiments, the surfactant comprises 2-[4-(2,4,4-trimethylpentan-2-yl)phenoxy]ethanol, also known as TRITON™ X-100. In some embodiments, the surfactant comprises a polysorbate surfactant, also known as a TWEEN® surfactant. Non-limiting examples of TWEEN® surfactants include polyoxyethylene (20) sorbitan monolaurate (polysorbate 20 or TWEEN® 20), polyoxyethylene (20) sorbitan monopalmitate (polysorbate 40 or TWEEN® 40), polyoxyethylene (20) sorbitan monostearate (polysorbate 60 or TWEEN® 60), and polyoxyethylene (20) sorbitan monooleate (polysorbate 80 or TWEEN® 80). In some embodiments, the surfactant comprises TRITON™ X-100, TWEEN® 20, TWEEN® 80, or a combination thereof. In some embodiments, the phenolic glycoside is hesperidin, and the polyphenol aglycone is hesperetin. In some embodiments, the method is a solid state catalysis reaction.

In some embodiments, the method of the present disclosure is performed for greater than about 1 hour, greater than about 2 hours, greater than about 3 hours, greater than about 4 hours, greater than about 5 hours, greater than about 6 hours, greater than about 7 hours, greater than about 8 hours, greater than about 9 hours, greater than about 10 hours, greater than about 12 hours, greater than about 14 hours, greater than about 16 hours, greater than about 18 hours, greater than about 20 hours, greater than about 22 hours, or greater than about 24 hours. In some embodiments, the method is performed for about 1 hour to about 48 hours, or about 2 hours to about 44 hours, or about 3 hours to about 40 hours, or about 4 hours to about 36 hours, or about 5 hours to about 32 hours, or about 6 hours to about 28 hours, or about 6 hours to about 24 hours, or about 7 hours to about 22 hours, or about 8 hours to about 20 hours, or about 9 hours to about 18 hours, or about 10 hours to about 16 hours, or about 12 hours to about 14 hours. In some embodiments, the phenolic glycoside is hesperidin, and the polyphenol aglycone is hesperetin. In some embodiments, the method is a solid state catalysis reaction.

As discussed herein, the glycoside hydrolases provided herein have high stability over long reaction times. In some embodiments, the catalytic activity of the glycoside hydrolase does not decrease by more than 5%, more than 10%, more than 15%, or more than 20% over a period of about 1, 2, 3, 4, 5, or 6 hours at about 40° C. to about 60° C., or about 45° C. to about 58° C., or about 48° C. to about 55° C., or about 52° C. In some embodiments, the phenolic glycoside is hesperidin, and the polyphenol aglycone is hesperetin. In some embodiments, the method is a solid state catalysis reaction.

In some embodiments, the method of the present disclosure is performed at about 35° C. to about 70° C., or about 37° C. to about 65° C., or about 40° C. to about 60° C., or about 42° C. to about 58° C., or about 45° C. to about 56° C., or about 48° C. to about 55° C., or about 50° C. to about 54° C., or about 51° C. to about 53° C. In some embodiments, the method is performed at about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some embodiments, the phenolic glycoside is hesperidin, and the polyphenol aglycone is hesperetin. In some embodiments, the method is a solid state catalysis reaction.

In some embodiments, the method of the present disclosure is performed at a pH of about 3.5 to about 7, or about 3.7 to about 6.5, or about 4 to about 6, or about 4.5 to about 5.8, or about 5 to about 5.6, or about 5.2 to about 5.5, or about 5.3 to about 5.4. In some embodiments, the method is performed at a pH of about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6. In some embodiments, the phenolic glycoside is hesperidin, and the polyphenol aglycone is hesperetin. In some embodiments, the method is a solid state catalysis reaction. It will be understood by one of ordinary skill in the art that in a solid state catalysis reaction described herein, the indicated pH refers to the pH of the aqueous solution in which the glycoside hydrolase is solubilized.

In some embodiments, the method of the present disclosure is performed at a pH of about 4, and at a temperature and time period according to Table 2 or Table 3. In some embodiments, the method is performed at a pH of about 4.5, and at a temperature and time period according to Table 2 or Table 3. In some embodiments, the method is performed at a pH of about 4.6, and at a temperature and time period according to Table 2 or Table 3. In some embodiments, the method is performed at a pH of about 4.7, and at a temperature and time period according to Table 2 or Table 3. In some embodiments, the method is performed at a pH of about 4.8, and at a temperature and time period according to Table 2 or Table 3. In some embodiments, the method is performed at a pH of about 4.9, and at a temperature and time period according to Table 2 or Table 3. In some embodiments, the method is performed at a pH of about 5, and at a temperature and time period according to Table 2 or Table 3. In some embodiments, the method is performed at a pH of about 5.1, and at a temperature and time period according to Table 2 or Table 3. In some embodiments, the method is performed at a pH of about 5.2, and at a temperature and time period according to Table 2 or Table 3. In some embodiments, the method is performed at a pH of about 5.3, and at a temperature and time period according to Table 2 or Table 3. In some embodiments, the method is performed at a pH of about 5.4, and at a temperature and time period according to Table 2 or Table 3. In some embodiments, the method is performed at a pH of about 5.5, and at a temperature and time period according to Table 2 or Table 3. In some embodiments, the method is performed at a pH of about 5.6, and at a temperature and time period according to Table 2 or Table 3. In some embodiments, the method is performed at a pH of about 5.7, and at a temperature and time period according to Table 2 or Table 3. In some embodiments, the method is performed at a pH of about 5.8, and at a temperature and time period according to Table 2 or Table 3. In some embodiments, the method is performed at a pH of about 5.9, and at a temperature and time period according to Table 2 or Table 3. In some embodiments, the method is performed at a pH of about 6, and at a temperature and time period according to Table 2 or Table 3. In some embodiments, the phenolic glycoside is hesperidin, and the polyphenol aglycone is hesperetin. In some embodiments, the method is a solid state catalysis reaction.

TABLE 2
Reaction Conditions - Temperatures and Time Ranges

| Temp | Time range (hours) | | | | | | | | |
|---|---|---|---|---|---|---|---|---|---|
| 40° C. | 1-48 | 2-44 | 3-40 | 4-36 | 5-32 | 6-28 | 6-24 | 7-22 | 8-20 |
| 41° C. | 1-48 | 2-44 | 3-40 | 4-36 | 5-32 | 6-28 | 6-24 | 7-22 | 8-20 |
| 42° C. | 1-48 | 2-44 | 3-40 | 4-36 | 5-32 | 6-28 | 6-24 | 7-22 | 8-20 |
| 43° C. | 1-48 | 2-44 | 3-40 | 4-36 | 5-32 | 6-28 | 6-24 | 7-22 | 8-20 |
| 44° C. | 1-48 | 2-44 | 3-40 | 4-36 | 5-32 | 6-28 | 6-24 | 7-22 | 8-20 |
| 45° C. | 1-48 | 2-44 | 3-40 | 4-36 | 5-32 | 6-28 | 6-24 | 7-22 | 8-20 |
| 46° C. | 1-48 | 2-44 | 3-40 | 4-36 | 5-32 | 6-28 | 6-24 | 7-22 | 8-20 |
| 47° C. | 1-48 | 2-44 | 3-40 | 4-36 | 5-32 | 6-28 | 6-24 | 7-22 | 8-20 |
| 48° C. | 1-48 | 2-44 | 3-40 | 4-36 | 5-32 | 6-28 | 6-24 | 7-22 | 8-20 |
| 49° C. | 1-48 | 2-44 | 3-40 | 4-36 | 5-32 | 6-28 | 6-24 | 7-22 | 8-20 |
| 50° C. | 1-48 | 2-44 | 3-40 | 4-36 | 5-32 | 6-28 | 6-24 | 7-22 | 8-20 |
| 51° C. | 1-48 | 2-44 | 3-40 | 4-36 | 5-32 | 6-28 | 6-24 | 7-22 | 8-20 |
| 52° C. | 1-48 | 2-44 | 3-40 | 4-36 | 5-32 | 6-28 | 6-24 | 7-22 | 8-20 |
| 53° C. | 1-48 | 2-44 | 3-40 | 4-36 | 5-32 | 6-28 | 6-24 | 7-22 | 8-20 |
| 54° C. | 1-48 | 2-44 | 3-40 | 4-36 | 5-32 | 6-28 | 6-24 | 7-22 | 8-20 |
| 55° C. | 1-48 | 2-44 | 3-40 | 4-36 | 5-32 | 6-28 | 6-24 | 7-22 | 8-20 |
| 56° C. | 1-48 | 2-44 | 3-40 | 4-36 | 5-32 | 6-28 | 6-24 | 7-22 | 8-20 |
| 57° C. | 1-48 | 2-44 | 3-40 | 4-36 | 5-32 | 6-28 | 6-24 | 7-22 | 8-20 |
| 58° C. | 1-48 | 2-44 | 3-40 | 4-36 | 5-32 | 6-28 | 6-24 | 7-22 | 8-20 |
| 59° C. | 1-48 | 2-44 | 3-40 | 4-36 | 5-32 | 6-28 | 6-24 | 7-22 | 8-20 |
| 60° C. | 1-48 | 2-44 | 3-40 | 4-36 | 5-32 | 6-28 | 6-24 | 7-22 | 8-20 |

TABLE 3
Reaction Conditions - Times and Temperature Ranges

| Time | | Temperature range (° C.) | | | | | | | | |
|---|---|---|---|---|---|---|---|---|---|---|
| 1 | hr | 35-70 | 37-65 | 40-60 | 42-58 | 45-56 | 48-55 | 50-54 | 51-53 | 52 |
| 2 | hrs | 35-70 | 37-65 | 40-60 | 42-58 | 45-56 | 48-55 | 50-54 | 51-53 | 52 |
| 3 | hrs | 35-70 | 37-65 | 40-60 | 42-58 | 45-56 | 48-55 | 50-54 | 51-53 | 52 |
| 4 | hrs | 35-70 | 37-65 | 40-60 | 42-58 | 45-56 | 48-55 | 50-54 | 51-53 | 52 |
| 5 | hrs | 35-70 | 37-65 | 40-60 | 42-58 | 45-56 | 48-55 | 50-54 | 51-53 | 52 |
| 6 | hrs | 35-70 | 37-65 | 40-60 | 42-58 | 45-56 | 48-55 | 50-54 | 51-53 | 52 |
| 7 | hrs | 35-70 | 37-65 | 40-60 | 42-58 | 45-56 | 48-55 | 50-54 | 51-53 | 52 |
| 8 | hrs | 35-70 | 37-65 | 40-60 | 42-58 | 45-56 | 48-55 | 50-54 | 51-53 | 52 |
| 9 | hrs | 35-70 | 37-65 | 40-60 | 42-58 | 45-56 | 48-55 | 50-54 | 51-53 | 52 |
| 10 | hrs | 35-70 | 37-65 | 40-60 | 42-58 | 45-56 | 48-55 | 50-54 | 51-53 | 52 |
| 11 | hrs | 35-70 | 37-65 | 40-60 | 42-58 | 45-56 | 48-55 | 50-54 | 51-53 | 52 |
| 12 | hrs | 35-70 | 37-65 | 40-60 | 42-58 | 45-56 | 48-55 | 50-54 | 51-53 | 52 |
| 14 | hrs | 35-70 | 37-65 | 40-60 | 42-58 | 45-56 | 48-55 | 50-54 | 51-53 | 52 |
| 16 | hrs | 35-70 | 37-65 | 40-60 | 42-58 | 45-56 | 48-55 | 50-54 | 51-53 | 52 |
| 18 | hrs | 35-70 | 37-65 | 40-60 | 42-58 | 45-56 | 48-55 | 50-54 | 51-53 | 52 |
| 20 | hrs | 35-70 | 37-65 | 40-60 | 42-58 | 45-56 | 48-55 | 50-54 | 51-53 | 52 |
| 22 | hrs | 35-70 | 37-65 | 40-60 | 42-58 | 45-56 | 48-55 | 50-54 | 51-53 | 52 |
| 24 | hrs | 35-70 | 37-65 | 40-60 | 42-58 | 45-56 | 48-55 | 50-54 | 51-53 | 52 |
| 26 | hrs | 35-70 | 37-65 | 40-60 | 42-58 | 45-56 | 48-55 | 50-54 | 51-53 | 52 |
| 28 | hrs | 35-70 | 37-65 | 40-60 | 42-58 | 45-56 | 48-55 | 50-54 | 51-53 | 52 |
| 30 | hrs | 35-70 | 37-65 | 40-60 | 42-58 | 45-56 | 48-55 | 50-54 | 51-53 | 52 |

In some embodiments, the percent conversion of the phenolic glycoside to the polyphenol aglycone by the glycoside hydrolase described herein is at least 20%. As used herein, “percent conversion” or “% conversion” refers to the percentage of the phenolic glycoside substrate is converted to the polyphenol aglycone product in a reaction. In some embodiments, the percent conversion of the phenolic glycoside to the polyphenol aglycone is at least 25%, at least 30%, at least 40%, at least 45%, at least 50%, at least 55%, at least 60%, at least 65%, at least 70%, at least 75%, at least 80%, at least 85%, or at least 95%. In some embodiments, the method is performed over a period of about 6 to about 24 hours at about 40° C. to about 60° C., and about pH 4 to about pH 6. In some embodiments, the phenolic glycoside is hesperidin, and the polyphenol aglycone is hesperetin. In some embodiments, the method is a solid state catalysis reaction.

In some embodiments, the percent conversion of the phenolic glycoside to the polyphenol aglycone is at least 40%, at least 45%, at least 50%, at least 55%, at least 60%, at least 65%, at least 70%, at least 75%, at least 80%, at least 85%, or at least 95%, when the method is performed over a period of about 8 to about 20 hours at about 48° C. to about 55° C., and about pH 4.5 to about pH 5.8. In some embodiments, the percent conversion of the phenolic glycoside to the polyphenol aglycone is at least 40%, at least 45%, at least 50%, at least 55%, at least 60%, at least 65%, at least 70%, at least 75%, at least 80%, at least 85%, or at least 95%, when the method is performed over a period of about 7 to about 22 hours at about 48° C. to about 55° C., and about pH 5 to about pH 5.6. In some embodiments, the percent conversion of the phenolic glycoside to the polyphenol aglycone is at least 40%, at least 45%, at least 50%, at least 55%, at least 60%, at least 65%, at least 70%, at least 75%, at least 80%, at least 85%, or at least 95%, when the method is performed over a period of about 8 to about 20 hours at about 50° C. to about 54° C., and about pH 5.2 to about pH 5.5. In some embodiments, the percent conversion of the phenolic glycoside to the polyphenol aglycone is at least 40%, at least 45%, at least 50%, at least 55%, at least 60%, at least 65%, at least 70%, at least 75%, at least 80%, at least 85%, or at least 95%, when the method is performed over a period of about 18 hours at about 52° C., and about pH 5.4. In some embodiments, the phenolic glycoside is hesperidin, and the polyphenol aglycone is hesperetin. In some embodiments, the method is a solid state catalysis reaction.

In some embodiments, the percent conversion of the phenolic glycoside to the polyphenol aglycone by the glycoside hydrolase comprising the first, second, third, fourth, fifth, and optionally sixth regions described herein, has a percent conversion rate that is at least 1.5-fold, at least 2-fold, at least 3-fold, at least 4-fold, at least 5-fold, at least 6-fold, least 7-fold, at least 8-fold, at least 9-fold, at least 10-fold, at least 15-fold, at least 20-fold, at least 25-fold, at least 30-fold, at least 40-fold, at least 50-fold, or at least 100-fold higher than the percent conversation rate by a glycoside hydrolase that does not comprise the first, second, third, fourth, fifth, and optionally sixth regions as described herein, e.g., a glycoside hydrolase from Acremonium sp. DSM 24697 as described in Weiz et al., Appl Microbiol Biotechnol (2019) 103:9493-9504. In some embodiments, the method is performed over a period of about 6 to about 24 hours at about 40° C. to about 60° C., and about pH 4 to about pH 6. In some embodiments, the phenolic glycoside is hesperidin, and the polyphenol aglycone is hesperetin. In some embodiments, the method is a solid state catalysis reaction.

### Compositions

In some embodiments, the present disclosure provides a composition comprising the polyphenol aglycone made by a method described herein. Exemplary polyphenol aglycones include, but are not limited to, naringenin, eriodictyol, apigenin, and hesperetin. In some embodiments, the polyphenol aglycone is hesperetin.

In some embodiments, the composition is a food or beverage composition. In some embodiments, the composition is a pharmaceutical composition. In some embodiments, the composition has reduced bitter taste as compared to an otherwise identical composition not comprising the polyphenol aglycone. In some embodiments, the composition has enhanced sweet taste as compared to an otherwise identical composition not comprising the polyphenol aglycone. In some embodiments, the composition is a cosmetic composition. In some embodiments, the composition has reduced microbial growth, reduced oxidation, and/or increased shelf life as compared to an otherwise identical composition that does not comprise the polyphenol aglycone. In some embodiments, the polyphenol aglycone is hesperetin.

All references cited herein, including patents, patent applications, papers, textbooks and the like, and the references cited therein, to the extent that they are not already, are hereby incorporated herein by reference in their entirety.

SEQUENCES
SEQ ID NO: 1:
HQEAII
SEQ ID NO 2:
DIHSLPGGLNGMGLGE
SEQ ID NO: 3:
PINEPVDNRDITKFGTP
SEQ ID NO: 4:
FRPVDYWAKHFAASTNIVFDVHNYYFAGRPT
SEQ ID NO: 5:
GSAYWTWKFFGNVPVDGEGTQGDYWNY
SEQ ID NO: 6:
KFPVFVGEWSIQAA
SEQ ID NO: 7:
CSDAKXXXXXXXXKFPVFVGEWSIQAA
SEQ ID NO: 8:
CSDAKDIVSTASPKFPVFVGEWSIQAA
SEQ ID NO: 9:
APPSNYLNWKTFKANGVNVGGWLHQEAVIDPTWWNQYAPGTPDEW
DFCARLKSQCGPILEQRYGSYITTKDIDTMAAAGINVIRVPTGYN
AWVTVPGSQLYSGNQARFLRTISDYAIKKHGIHVILDIHSLPGGL
NGMGLGEKEGNYGWFQNQTALDYSYQAVDAAIKFIQGSDVPQGFT
LAPINEPVDNRDFTKFGTPEALTEEGAAWVLEYFQGVISRVEKAN
PKIPIMLQGGFRSVDFWAKYFAVSTNLVFDVHNYYFAGRPTTSQK
LPEFICTDAKNTVSSTSLKFPVFVGEWSIQAAANNTFASRARNLN
TGLKAWATYTQGSAYWTWKFFGNEPVDGEGTQGDYWNYSDFVKMG
IIDPSSGVTCK
SEQ ID NO: 10:
LPPSTYLNWKTFKANGVNIGGWLHQEAVIDPKWWNQYAPGTPDEW
DFCAKLGSQCGPILEQRYSSFITTKDIDAMAKAGINVIRIPTGYN
AWVTVPGSQLYSGNQARFLRVISDYAIKKHGIHVILDIHSLPGGL
NGMGLGEKEGNYGWFQNQTALDYSYKAVNAAIKFIQESDVPQGFT
LAPINEPVDNRDITKFGTPEALSGEGAAWVLRYFQGVVSRVQKIN
PKIPIMLQGGFRPVDFWAKNFAANTNIVFDVHHYYFAGRPATSQN
LPDLICTDAKSSAVTVEPKFPVFVGEWSIQATSDNNFSSRAINLN
AGLKAWSKYTRGSAYWTWKFFGNVPVDGEGTQGDYWNYSDFVKMA
IINPSTGISCK
SEQ ID NO: 11:
APPFNYLNWKTFKANGVNLGGWLHQEAVIDPKWWNQYAPGTPDEW
DFCAKLKSQCGPILEQRYGSYITTKDIDTIAAAGINVIRVPTGYN
AWVTVPGSQLYSGNQARFLRTLSDYAIKKHGIHVILDIHSLPGGL
NGMGLGEKEGNYGWFQNQTALDYSYKVVDAAIKFIQESDVPQGFT
LAPINEPVDNRDFTKFGTPEALSEEGAAWVLKYFQGVISRVEKTN
PKIPIMLQGGFRPVDFWAKYFTASTNLVFDVHDYYFAGRPTTSQN
LPEFICTDAKNTVNTTPPKFPVFVGEWSIQAASNNTFASRARNLN
TGLKAWATYTQGSAYWTWKFFGNEPVNGEGTQGDYWNYSDFVKMG
LINPSSGVSCK
SEQ ID NO: 12:
APPSNYLNWKTFKANGVNLGGWLHQEAIIDPTWWNQYAPGMPDEW
DFCAKLKSQCGPVLEQRYGSYITAKDIDTMAAAGINVIRVPTGYN
AWVTVPGSQLYSGNQARYLRAISDYAIKKHGIYVILDIHSLPGGL
NGMGLGEKEGNYGWFQNQTALEYSYKAVDAALKFIQESDVPQGFT
LAPINEPVDNRDITKFGTPDALSDQGAAWVLQYFQGVISRVEKVN
PKIPIMLQGGFRPVDYWAKHFAASTNIVFDVHNYYFAGRPTTSQN
LAELICSDAKDIVSTASPKFPVFVGEWSIQAATNNTFASRARNLN
TGLKAWTAYTRGSAYWTWKFFGNVPVDGEGTQGDYWNYSDFVKMG
IISPSSEVTCK
SEQ ID NO: 13:
MRFPSIFTAVLFAASSALAAPVNTTTEDETAQIPAEAVIGYSDLE
GDFDVAVLPFSNSTNNGLLFINTTIASIAAKEEGVSLEKREAEA
SEQ ID NO: 14 (from U.S. Pat. No. 7,998,721):
STYLNWTTFNAVGANLGGWLVQESTIDTTWWAQYSGGAVDEWGLC
AYQGSQCGPVLERRYATWITTADIDTLGAAGVNVLRIPTTYAAWV
KVPGSQLYHGNQQSFLASISSYAINKYGMHIIIDIHSLPGGVNGF
PFGEAYGHYGWENNQTALKYSLEAVDAAISFIQNSNSPQSYTLAP
INEPVDVEDLSLFGTPYCLTDDGAAYLASYMHQVIAKVEAVNSEI
PIMFQGSFKGEAYWSSTFTSDTNLVFDIHNYYFEGRAASSTNVTQ
LICADAVTSAGDGKFPTFVGEWSIQTQIANNFASRAKILETGLAA
WKKYTRGSAYWTTKFTGNATVDGEGTQADYWNYETFINLGYTKST
SAAVPC

### EXAMPLES

### Example 1. Plasmid Transformation and Integration of Glycoside Hydrolase into Pichia

Genes encoding the glycoside hydrolases according to embodiments herein, e.g., SEQ ID NOs: 9-12, were synthesized and cloned into a plasmid containing a Zeocin resistance cassette for proper selection, as well as the alpha mate factor secretion tag from Saccharomyces cerevisiae for secretion into the extracellular medium. The resulting plasmid was transformed into previously prepared electrocompetent cells from Pichia pastoris (now Komagataella phaffi), and resulting transformants were selected on YPD agar plates supplemented with Zeocin. The highest protein producing strain was selected and grown on YPD+Zeocin. Glycerol stocks were made from this culture.

### Example 2. Protein Expression in Shake Flask

For protein expression, cells were first streaked onto a BMG-agar (YNB, biotin, glycerol) plate supplemented with zeocin and grown at 30° C. for 72 hours. A single colony was inoculated into 50 mL of BMG media (YNB, potassium phosphate, biotin, glycerol) and grown at 30° C. for 24 hours. The next day, 500 mL of BMM media (YNB, biotin, potassium phosphate, methanol) were inoculated with the initial culture to a final OD of 1, and the culture was grown at 30° C. under agitation for 72 hours. An addition of 0.5% methanol was necessary every 24 hours for continuous protein induction.

After 72 hours, the culture was spun down, the cells were discarded, and the supernatant was filtered through a 10 kDa filter to remove any small molecules and salts. The resulting protein was concentrated to 2 g/L as determined by BCA assay.

### Example 3. Protein Expression in Fermentation

For fermentation protein expression, cells were streaked onto a BMG-agar plate supplemented with zeocin and grown at 30° C. for 72 hours. A single colony was inoculated into 50 mL of BMG media and grown at 30° C. for 24 hours. After 24 hours, a liter of BMM media was inoculated from the stage I culture to an OD of 0.06 and grown at 24 hours for approximately 16 hours. After this time, the final OD of the stage II culture was 8. A bioreactor containing basal salt media supplemented with 4% glycerol was inoculated with 10% working volume from the stage II culture. Dissolved oxygen was kept at 30% during the initial phase and the agitation maintained at a minimum of 600 rpm. pH was controlled with ammonium hydroxide and maintained at 6 throughout the entire fermentation. A feed of 50% glycerol was initiated after cells reached an OD of about 30 and kept for about 2 to 3 hours until the OD reached around 100. After this time, the glycerol feed was stopped, and a methanol feed was initiated with progressive increases in the first few hours. The methanol feed was then kept steady for about 50 hours.

After almost 72 hours in the bioreactors, cells were spun down in a centrifuge and the biomass was discarded. The resulting supernatant was passed through a 0.2 uM TFF filter. The filtrate, which contains the protein, was then passed through a low molecular weight cut-off TFF filter to remove any small molecules and salts.

### Example 4. Production of Hesperetin

To produce hesperetin, an appropriate amount of concentrated protein produced according to the previous Examples, was supplemented with surfactant and hesperidin. The reaction was incubated at 52° C. under optimal agitation. Conversion of hesperidin into hesperetin was analyzed using HPLC.

The reaction was subsequently filtered, and the filtrate was treated and washed with 0.2% SDS at pH 5. The sample was filtered again and the filtrate was dried under vacuum oven and the purity was analyzed using HPLC.

### Example 5. Production of Hesperetin with Different Co-Solvents and Surfactants

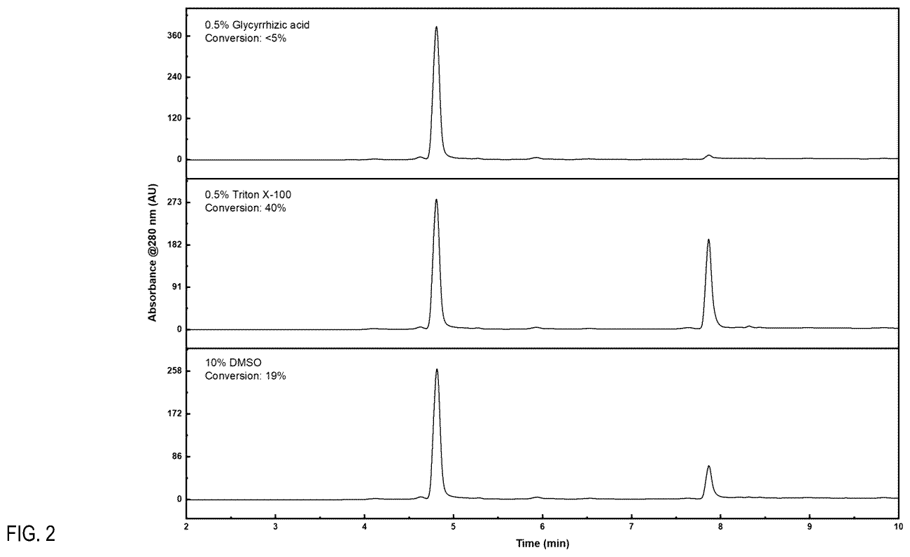

Hesperetin production was tested in the presence of different co-solvents and surfactants. The reactions were performed essentially as described in Example 4, except the 0.1% TRITON™ X-100 supplement was replaced with either 0.5% glycyrrhizic acid, 0.5% TRITON™ X-100, or 10% DMSO. The results are shown in FIG. 2 and summarized as follows:
0.5% Glycyrrhizic acid: <5% conversion
0.5% TRITON™ X-100: 40% conversion
10% DMSO: 19% conversion

Of the tested conditions, the highest hesperetin production was observed with TRITON™ X-100.

### Example 6. Production of Hesperetin by SEQ ID NO:12 and AoGH5

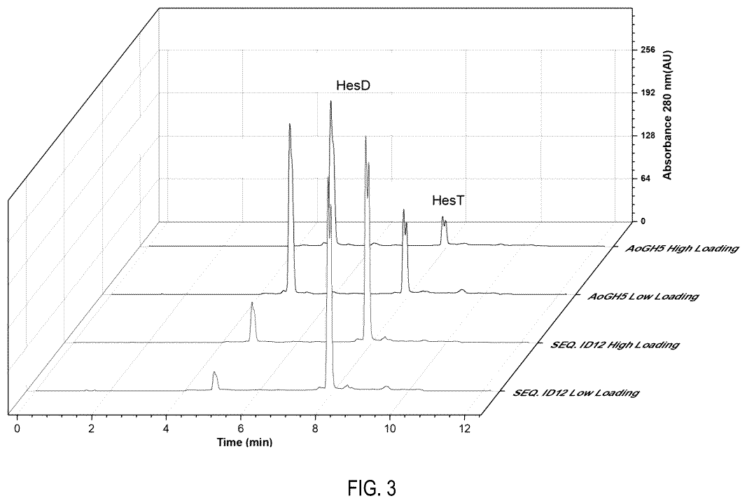

Hesperetin (“HesT”) production from hesperidin (“HesD”) was compared between the glycoside hydrolase of SEQ ID NO: 12 (“SEQ ID NO:12”), as described herein, and the glycoside hydrolase from Acremonium sp. DSM 24697, as described in Weiz et al., Appl Microbiol Biotechnol (2019) 103:9493-9504 (“AoGH5”). The reaction was conducted essentially as described in Example 4. The results in FIG. 3 show that AoGH5 exhibited a conversion rate of less than 50% at both high and low substrate loading conditions. In contrast, SEQ ID NO: 12 achieved a conversion rate exceeding 80% at both high and low substrate loading conditions, demonstrating a significantly higher efficiency in converting hesperidin to hesperetin as compared to AoGH5.

1. A composition comprising a glycoside hydrolase and one or both of hesperidin and hesperetin, wherein an active site of the glycoside hydrolase comprises:
a first region comprising at least 80%, at least 90%, or 100% identity to HQEAII (SEQ ID NO:1);
a second region comprising at least 80%, at least 90%, or 100% identity to DIHSLPGGLNGMGLGE (SEQ ID NO:2);
a third region comprising at least 80%, at least 90%, or 100% identity to PINEPVDNRDITKFGTP (SEQ ID NO:3);
a fourth region comprising at least 80%, at least 90%, or 100% identity to FRPVDYWAKHFAASTNIVFDVHNYYFAGRPT (SEQ ID NO:4); and
a fifth region comprising at least 80%, at least 90%, or 100% identity to GSAYWTWKFFGNVPVDGEGTQGDYWNY (SEQ ID NO:5).

2. (canceled)

3. (canceled)

4. The composition of claim 1, wherein the active site of the glycoside hydrolase further comprises a sixth region comprising at least 80%, at least 90%, or 100% identity to KFPVFVGEWSIQAA (SEQ ID NO:6) or CSDAKXXXXXXXXKFPVFVGEWSIQAA (SEQ ID NO:7).

5-7. (canceled)

8. The composition of claim 1, wherein the glycoside hydrolase comprises at least 60%, at least 70%, at least 80%, at least 90%, or 100% sequence identity to any one of SEQ ID NOs: 9-12.

9-12. (canceled)

13. The composition of claim 1, further comprising DMSO, a surfactant, or both.

14. The composition of claim 13, wherein the surfactant comprises TRITON X-100, TWEEN, or a combination thereof.

15. The composition of claim 1, wherein the glycoside hydrolase is capable of hydrolyzing the hesperidin.

16. The composition of claim 1, wherein the glycoside hydrolase is solubilized in an aqueous solution and the hesperidin and the hesperetin are in solid form.

17. A method of producing a polyphenol aglycone, comprising contacting a phenolic glycoside with a glycoside hydrolase, wherein an active site of the glycoside hydrolase comprises:
a first region comprising at least 80%, at least 90%, or 100% identity to HQEAII (SEQ ID NO:1);
a second region comprising at least 80%, at least 90%, or 100% identity to DIHSLPGGLNGMGLGE (SEQ ID NO:2);
a third region comprising at least 80%, at least 90%, or 100% identity to PINEPVDNRDITKFGTP (SEQ ID NO:3);
a fourth region comprising at least 80%, at least 90%, or 100% identity to FRPVDYWAKHFAASTNIVFDVHNYYFAGRPT (SEQ ID NO:4); and
a fifth region comprising at least 80%, at least 90%, or 100% identity to GSAYWTWKFFGNVPVDGEGTQGDYWNY (SEQ ID NO:5);
wherein the glycoside hydrolase hydrolyzes the phenolic glycoside to form the polyphenol aglycone.

18. The method of claim 17, wherein the glycoside hydrolase is solubilized in an aqueous solution, and the phenolic glycoside is in solid form upon contacting with the glycoside hydrolase.

19. The method of claim 17, wherein the phenolic glycoside comprises a structure of Formula I: 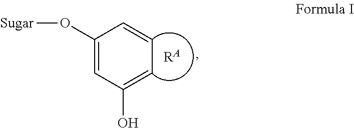
wherein the sugar is a monosaccharide or a disaccharide, and R^{A} is cycloalkyl or heterocyclyl optionally substituted with one or more oxo, alkyl, alkoxyl, alkenyl, alkynyl, hydroxyl, cycloalkyl, heterocyclyl, aryl, or heteroaryl, wherein the cycloalkyl, heterocyclyl, aryl, or heteroaryl is unsubstituted or substituted with one or more oxo, alkyl, alkoxyl, alkenyl, alkynyl, or hydroxyl.

20. The method of claim 17, wherein the phenolic glycoside is hesperidin and the polyphenol aglycone is hesperetin.

21. (canceled)

22. The method of claim 17, wherein:
(i) the active site of the glycoside hydrolase further comprises a sixth region comprising at least 80%, at least 90%, or 100% sequence identity to KFPVFVGEWSIQAA (SEQ ID NO: 6) or CSDAKXXXXXXXXKFPVFVGEWSIQAA (SEQ ID NO:7);
(ii) the glycoside hydrolase comprises at least 60%, at least 70%, at least 80%, at least 90%, or 100% sequence identity to any one of SEQ ID NOs: 9-12;
(iii) the contacting is in the presence of DMSO, a surfactant, or both;
(iv) the surfactant comprises TRITON X-100, TWEEN, or a combination thereof;
(v) the method is performed for about 6 hours to about 24 hours;
(vi) the method is performed at about 40° C. to about 60° C.;
(vii) the method is performed at about pH 4 to about pH 6; or
(viii) a percent conversion of the phenolic glycoside to the polyphenol aglycone is at least 40% or at least 80%; or
(ix) or any combination of (i)-(viii).

23-30. (canceled)

31. A composition comprising a polyphenol aglycone made by the method of claim 17.

32. The composition of claim 31, wherein the polyphenol aglycone is hesperetin.

33. A host cell capable of expressing a heterologous glycoside hydrolase, wherein an active site of the glycoside hydrolase comprises:
a first region comprising at least 80% identity to HQEAII (SEQ ID NO:1);
a second region comprising at least 80% identity to DIHSLPGGLNGMGLGE (SEQ ID NO:2);
a third region comprising at least 80% identity to PINEPVDNRDITKFGTP (SEQ ID NO: 3);
a fourth region comprising at least 80% identity to FRPVDYWAKHFAASTNIVFDVHNYYFAGRPT (SEQ ID NO:4); and
a fifth region comprising at least 80% identity to GSAYWTWKFFGNVPVDGEGTQGDYWNY (SEQ ID NO:5),
wherein the glycoside hydrolase is capable of hydrolyzing hesperidin to form hesperetin.

34. An expression system comprising the host cell of claim 33.

35-38. (canceled)

39. A nucleic acid encoding a glycosidic hydrolase, wherein an active site of the glycoside hydrolase comprises:
a first region comprising at least 80% or at least 90% identity to HQEAII (SEQ ID NO: 1);
a second region comprising at least 80% or at least 90% identity to DIHSLPGGLNGMGLGE (SEQ ID NO:2);
a third region comprising at least 80% or at least 90% identity to PINEPVDNRDITKFGTP (SEQ ID NO:3);
a fourth region comprising at least 80% or at least 90% identity to FRPVDYWAKHFAASTNIVFDVHNYYFAGRPT (SEQ ID NO:4); and
a fifth region comprising at least 80% or at least 90% identity to GSAYWTWKFFGNVPVDGEGTQGDYWNY (SEQ ID NO:5),
wherein the glycoside hydrolase is capable of hydrolyzing hesperidin to form hesperetin, and wherein the nucleic acid is operably linked to a heterologous regulatory element.

40. The nucleic acid of claim 39, wherein;
(i) the heterologous regulatory element is a promoter, optionally a constitutive or inducible promoter;
(ii) the nucleic acid is linked at its 5′ end to a sequence encoding an alpha-factor signal sequence, optionally wherein the alpha-factor signal sequence comprises SEQ ID NO: 13;
(iii) the active site of the glycoside hydrolase further comprises a sixth region comprising at least 80%, at least 90%, or 100% sequence identity to KFPVFVGEWSIQAA (SEQ ID NO: 6) or CSDAKXXXXXXXXKFPVFVGEWSIQAA (SEQ ID NO:7);
(iv) the glycoside hydrolase comprises at least 60%, at least 70%, at least 80%, at least 90%, or 100% sequence identity to any one of SEQ ID NOs: 9-12; or
(v) any combination of (i)-(iv).

41-46. (canceled)

47. A vector comprising the nucleic acid of claim 39.

48. The vector of claim 47, wherein the vector is a fungal expression vector, optionally a Pichia expression vector or a Trichoderma expression vector.


SEQUENCE LISTING (14 sequences)

SEQ ID NO: 1 - protein, 6 aa, synthetic construct
HQEAII

SEQ ID NO: 2 - protein, 16 aa, synthetic construct
DIHSLPGGLNGMGLGE

SEQ ID NO: 3 - protein, 17 aa, synthetic construct
PINEPVDNRDITKFGTP

SEQ ID NO: 4 - protein, 31 aa, synthetic construct
FRPVDYWAKHFAASTNIVFDVHNYYFAGRPT

SEQ ID NO: 5 - protein, 27 aa, synthetic construct
GSAYWTWKFFGNVPVDGEGTQGDYWNY

SEQ ID NO: 6 - protein, 14 aa, synthetic construct
KFPVFVGEWSIQAA

SEQ ID NO: 7 - protein, 27 aa, synthetic construct
CSDAKXXXXXXXXKFPVFVGEWSIQAA

SEQ ID NO: 8 - protein, 27 aa, synthetic construct
CSDAKDIVSTASPKFPVFVGEWSIQAA

SEQ ID NO: 9 - protein, 371 aa, unidentified
APPSNYLNWKTFKANGVNVGGWLHQEAVIDPTWWNQYAPGTPDEWDFCARLKSQCGPILEQRYGSYITTKDIDTMAAAGINVIRVPTGYNAWVTVPGSQLYSGNQARFLRTISDYAIKKHGIHVILDIHSLPGGLNGMGLGEKEGNYGWFQNQTALDYSYQAVDAAIKFIQGSDVPQGFTLAPINEPVDNRDFTKFGTPEALTEEGAAWVLEYFQGVISRVEKANPKIPIMLQGGFRSVDFWAKYFAVSTNLVFDVHNYYFAGRPTTSQKLPEFICTDAKNTVSSTSLKFPVFVGEWSIQAAANNTFASRARNLNTGLKAWATYTQGSAYWTWKFFGNEPVDGEGTQGDYWNYSDFVKMGIIDPSSGVTCK

SEQ ID NO: 10 - protein, 371 aa, unidentified
LPPSTYLNWKTFKANGVNIGGWLHQEAVIDPKWWNQYAPGTPDEWDFCAKLGSQCGPILEQRYSSFITTKDIDAMAKAGINVIRIPTGYNAWVTVPGSQLYSGNQARFLRVISDYAIKKHGIHVILDIHSLPGGLNGMGLGEKEGNYGWFQNQTALDYSYKAVNAAIKFIQESDVPQGFTLAPINEPVDNRDITKFGTPEALSGEGAAWVLRYFQGVVSRVQKINPKIPIMLQGGFRPVDFWAKNFAANTNIVFDVHHYYFAGRPATSQNLPDLICTDAKSSAVTVEPKFPVFVGEWSIQATSDNNFSSRAINLNAGLKAWSKYTRGSAYWTWKFFGNVPVDGEGTQGDYWNYSDFVKMAIINPSTGISCK

SEQ ID NO: 11 - protein, 371 aa, unidentified
APPFNYLNWKTFKANGVNLGGWLHQEAVIDPKWWNQYAPGTPDEWDFCAKLKSQCGPILEQRYGSYITTKDIDTIAAAGINVIRVPTGYNAWVTVPGSQLYSGNQARFLRTLSDYAIKKHGIHVILDIHSLPGGLNGMGLGEKEGNYGWFQNQTALDYSYKVVDAAIKFIQESDVPQGFTLAPINEPVDNRDFTKFGTPEALSEEGAAWVLKYFQGVISRVEKTNPKIPIMLQGGFRPVDFWAKYFTASTNLVFDVHDYYFAGRPTTSQNLPEFICTDAKNTVNTTPPKFPVFVGEWSIQAASNNTFASRARNLNTGLKAWATYTQGSAYWTWKFFGNEPVNGEGTQGDYWNYSDFVKMGLINPSSGVSCK

SEQ ID NO: 12 - protein, 371 aa, unidentified
APPSNYLNWKTFKANGVNLGGWLHQEAIIDPTWWNQYAPGMPDEWDFCAKLKSQCGPVLEQRYGSYITAKDIDTMAAAGINVIRVPTGYNAWVTVPGSQLYSGNQARYLRAISDYAIKKHGIYVILDIHSLPGGLNGMGLGEKEGNYGWFQNQTALEYSYKAVDAALKFIQESDVPQGFTLAPINEPVDNRDITKFGTPDALSDQGAAWVLQYFQGVISRVEKVNPKIPIMLQGGFRPVDYWAKHFAASTNIVFDVHNYYFAGRPTTSQNLAELICSDAKDIVSTASPKFPVFVGEWSIQAATNNTFASRARNLNTGLKAWTAYTRGSAYWTWKFFGNVPVDGEGTQGDYWNYSDFVKMGIISPSSEVTCK

SEQ ID NO: 13 - protein, 89 aa, unidentified
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 14 - protein, 366 aa, unidentified
STYLNWTTFNAVGANLGGWLVQESTIDTTWWAQYSGGAVDEWGLCAYQGSQCGPVLERRYATWITTADIDTLGAAGVNVLRIPTTYAAWVKVPGSQLYHGNQQSFLASISSYAINKYGMHIIIDIHSLPGGVNGFPFGEAYGHYGWFNNQTALKYSLEAVDAAISFIQNSNSPQSYTLAPINEPVDVEDLSLFGTPYCLTDDGAAYLASYMHQVIAKVEAVNSEIPIMFQGSFKGEAYWSSTFTSDTNLVFDIHNYYFEGRAASSTNVTQLICADAVTSAGDGKFPTFVGEWSIQTQIANNFASRAKILETGLAAWKKYTRGSAYWTTKFTGNATVDGEGTQADYWNYETFINLGYTKSTSAAVPC